(19) United States
(12) Patent Application Publication
(10) Pub. No.: US 2023/0364146 A1
(43) Pub. Date: Nov. 16, 2023

(54) COMPOSITIONS AND METHODS FOR CD30 GENE MODIFICATION

(71) Applicant: VOR BIOPHARMA INC., Cambridge, MA (US)

(72) Inventors: John Lydeard, Cambridge, MA (US); Mark Jones, Cambridge, MA (US); Michael Pettiglio, Cambridge, MA (US); Tirtha Chakraborty, Cambridge, MA (US)

(73) Assignee: VOR BIOPHARMA INC., Cambridge, MA (US)

(21) Appl. No.: 18/029,158

(22) PCT Filed: Sep. 30, 2021
(86) PCT No.: PCT/US2021/052892

§ 371 Date: Mar. 29, 2023

Related U.S. Application Data
(60) Provisional application No. 63/085,194, filed on Sep. 30, 2020.

(51) Int. Cl. A61K 35/28; C12N 15/113; A61P 35/00; C12N 5/0789; C12N 5/0783; A61K 35/17

(52) U.S. Cl. CPC A61K 35/28; A61K 35/17; A61P 35/00; C12N 5/0636; C12N 5/0647; C12N 15/1138; A61K 2039/505

(57) ABSTRACT

Provided herein are gRNA comprising a targeting domain that targets CD30, which may be used, for example, to make modifications in cells. Also provided herein are methods of genetically engineered cell having a modification (e.g., insertion or deletion) in the CD30 gene and methods involving administering such genetically engineered cells to a subject, such as a subject having a hematopoietic malignancy.

### RELATED APPLICATION

This application claims the benefit under 35 U.S.C. 119(e) of U.S. Provisional Application No. 63/085,194, filed Sep. 30, 2020, which is incorporated by reference herein in its entirety.

### BACKGROUND

When a subject is administered an immunotherapy targeting an antigen associated with a disease or condition, e.g., an anti-cancer CAR-T therapy, the therapy can deplete not only the pathological cells intended to be targeted, but also non-pathological cells that may express the targeted antigen. This “on-target, off-disease” effect has been reported for some CAR-T therapeutics, e.g., those targeting CD19 or CD33. If the targeted antigen is expressed on the surface of cells required for survival or the subject, or on the surface of cells the depletion of which is of significant detriment to the health of the subject, the subject may not be able to receive the immunotherapy, or may have to face severe side effects once administered such a therapy. In other instances, it may be desirable to administer an immunotherapy targeting an antigen that is expressed on the immune effector cells that constitute the immunotherapy, e.g., on the surface of CAR-T cells, which may result in fratricide and render the respective therapeutics ineffective or virtually impossible to produce.

### SUMMARY

Some aspects of this disclosure describe compositions, methods, strategies, and treatment modalities that address the detrimental on-target, off-disease effects of certain immunotherapeutic approaches, e.g., of immunotherapeutics comprising lymphocyte effector cells targeting a specific antigen in a subject in need thereof, such a s CAR-T cells or CAR-NK cells.

Aspects of the present disclosure provide guide RNAs (gRNA) comprising a targeting domain comprising a sequence described in Tables 1-4. In some aspects, the gRNA comprises a targeting domain, wherein the targeting domain comprises a sequence of any one of SEQ ID NOs: 55-81 and 102-111. In some embodiments, the gRNA comprises a first complementarity domain, a linking domain, a second complementarity domain which is complementary to the first complementarity domain, and a proximal domain. In some embodiments, the gRNA is a single guide RNA (sgRNA).

In some embodiments, the gRNA comprises one or more nucleotide residues that are chemically modified. In some embodiments, the gRNA comprises one or more nucleotide residues that comprise a 2′O-methyl moiety. In some embodiments, the gRNA comprises one or more nucleotide residues that comprise a phosphorothioate. In some embodiments, the gRNA comprises one or more nucleotide residues that comprise a thioPACE moiety.

Aspects of the present disclosure provide methods of producing a genetically engineered cell, comprising: providing a cell, and contacting the cell with (i) any of the gRNAs described herein or a gRNA targeting a targeting domain targeted by any of the gRNAs described herein; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell. In some embodiments, the contacting comprises introducing (i) and (ii) into the cell in the form of a pre-formed ribonucleoprotein (RNP) complex. In some embodiments, the contacting comprises introducing (i) and/or (ii) into the cell in the form of a nucleic acid encoding the gRNA of (i) and/or the RNA-guided nuclease of (ii). In some embodiments, the nucleic acid encoding the gRNA of (i) and/or the RNA-guided nuclease of (ii) is an RNA, preferably an mRNA or an mRNA analog. In some embodiments, the ribonucleoprotein complex is introduced into the cell via electroporation.

In some embodiments, the RNA-guided nuclease is a CRISPR/Cas nuclease. In some embodiments, the CRISPR/Cas nuclease is a Cas9 nuclease. In some embodiments, the CRISPR/Cas nuclease is an spCas nuclease. In some embodiments, the Cas nuclease in an saCas nuclease. In some embodiments, the CRISPR/Cas nuclease is a Cpfl nuclease.

In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte.

Aspects of the present disclosure provide genetically engineered cells obtained by any of the methods described herein. Aspects of the present disclosure provide cell populations comprising the genetically engineered cells described herein.

Aspects of the present disclosure provide cell populations comprising a genetically engineered cell, wherein the genetically engineered cell comprises a genomic modification that consists of an insertion or deletion immediately proximal to a site cut by an RNA-guided nuclease when bound to a gRNA comprising a targeting domain as described in any of Tables 1-4. In some embodiments, wherein the genomic modification is an insertion or deletion generated by a non-homologous end joining (NHEJ) event. In some embodiments, wherein the genomic modification is an insertion or deletion generated by a homology-directed repair (HDR) event. In some embodiments, the genomic modification results in a loss-of function of CD30 in a genetically engineered cell harboring such a genomic modification. In some embodiments, the genomic modification results in a reduction of expression of CD30 to less than 25%, less than 20% less than 10% less than 5% less than 2% less than 1%, less than 0.1%, less than 0.01%, or less than 0.001% as compared to the expression level of CD30 in wild-type cells of the same cell type that do not harbor a genomic modification of CD30. In some embodiments, the genetically engineered cell is a hematopoietic stem or progenitor cell.

In some embodiments, the genetically engineered cell is an immune effector cell. In some embodiments, the genetically engineered cell is a T-lymphocyte. In some embodiments, the immune effector cell expresses a chimeric antigen receptor (CAR). In some embodiments, the CAR targets CD30.

In some embodiments, the cell population is characterized by the ability to engraft CD30-edited hematopoietic stem cells in the bone marrow of a recipient and to generate differentiated progeny of all blood lineage cell types in the recipient. In some embodiments, the cell population is characterized by the ability to engraft CD30-edited hematopoietic stem cells in the bone marrow of a recipient at an efficiency of at least 50%. In some embodiments, the cell population is characterized by the ability to engraft CD30-edited hematopoietic stem cells in the bone marrow of a recipient at an efficiency of at least 60%. In some embodiments, the cell population is characterized by the ability to engraft CD30-edited hematopoietic stem cells in the bone marrow of a recipient at an efficiency of at least 70%. In some embodiments, the cell population is characterized by the ability to engraft CD30-edited hematopoietic stem cells in the bone marrow of a recipient at an efficiency of at least 80%. In some embodiments, the cell population is characterized by the ability to engraft CD30-edited hematopoietic stem cells in the bone marrow of a recipient at an efficiency of at least 90%. In some embodiments, the cell population comprises CD30-edited hematopoietic stem cells that are characterized by a differentiation potential that is equivalent to the differentiation potential of non-edited hematopoietic stem cells.

Aspects of the present disclosure provide methods comprising administering to a subject in need thereof any of the genetically engineered cells described herein or any of the cell populations described herein. In some embodiments, the subject has or has been diagnosed with a hematopoietic malignancy. In some embodiments, the method further comprises administering to the subject an effective amount of an agent that targets CD30, wherein the agent comprises an antigen-binding fragment that binds CD30.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 is a model showing the structure of TNF receptor associated factor 2 in complex with a human CD30 peptide, obtained from www.rcsb.org/structure/1D01.

FIG. 2 is a schematic showing the location of exemplary guide RNAs described herein relative to the human CD30/TNFSF8 gene.

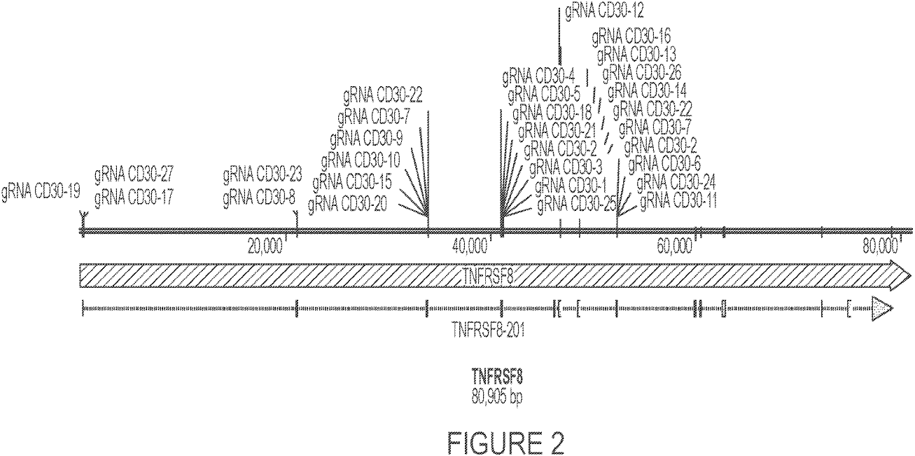

### DETAILED DESCRIPTION

Some aspects of this disclosure provide compositions, methods, strategies, and treatment modalities related to genetically modified cells, e.g., hematopoietic cells, that are deficient in the expression of an antigen targeted by a therapeutic agent, e.g., an immunotherapeutic agent. The genetically modified cells provided herein are useful, for example, to mitigate, or avoid altogether, certain undesired effects, for example, any on-target, off-disease cytotoxicity, associated with certain immunotherapeutic agents.

Such undesired effects associated with certain immunotherapeutic agents may occur, for example, when healthy cells within a subject in need of an immunotherapeutic intervention express an antigen targeted by an immunotherapeutic agent. For example, a subject may be diagnosed with a malignancy associated with an elevated level of expression of a specific antigen, which is not typically expressed in healthy cells, but may be expressed at relatively low levels in a subset of non-malignant cells within the subject. Administration of an immunotherapeutic agent, e.g., a CAR-T cell therapeutic or a therapeutic antibody or antibody-drug-conjugate (ADC) targeting the antigen, to the subject may result in efficient killing of the malignant cells, but may also result in ablation of non-malignant cells expressing the antigen in the subject. This on-target, off-disease cytotoxicity can result in significant side effects and, in some cases, abrogate the use of an immunotherapeutic agent altogether.

The compositions, methods, strategies, and treatment modalities provided herein address the problem of on-target, off-disease cytotoxicity of certain immunotherapeutic agents. For example, some aspects of this disclosure provide genetically engineered cells harboring a modification in their genome that results in a lack of expression of an antigen, or a specific form of that antigen, targeted by an immunotherapeutic agent. Such genetically engineered cells, and their progeny, are not targeted by the immunotherapeutic agent, and thus not subject to any cytotoxicity effected by the immunotherapeutic agent. Such cells can be administered to a subject receiving an immunotherapeutic agent targeting the antigen, e.g., in order to replace healthy cells that may have been targeted and killed by the cytotherapeutic agent, and/or in order to provide a population of cells that is resistant to targeting by the cytotherapeutic agent. For example, if healthy hematopoietic cells in the subject express the antigen, genetically engineered hematopoietic cells provided herein, e.g., genetically engineered hematopoietic stem or progenitor cells, may be administered to the subject that do not express the antigen, and thus are not targeted by the cytotherapeutic agent. Such hematopoietic stem or progenitor cells are able to re-populate the hematopoietic niche in the subject and their progeny can reconstitute the various hematopoietic lineages, including any that may have been ablated by the cytotherapeutic agent.

CD30, also referred to as tumor necrosis factor superfamily member 8 (TNFRSF8), is a 120 kDa transmembrane glycoprotein receptor for TNFSF8/CD30L. Stimulation of CD30, which leads to activation of the TRAF5 and TRAF2 signal transduction pathways, has been shown to lead to NF-κB activation and regulate cellular growth and transformation of activated lymphoblasts and be involved in increasing susceptibility of the cell to apoptosis. See, Sauder et al. Hematol. Oncol. Clin. North Am. (2017) 31(2): 317-334. Without wishing to be bound by any particular theory, CD30 is thought to play a role in immunosurveillance and cross-talk between T cells and B cells.

The gene encoding human CD30 contains 8 exons and is located on chromosome 1. The protein has been reported to be present in several isoforms due to alternative splicing. See, e.g., Wong et al. Blood (2018) 132 (Supplement 1): 5311.

In addition to its normal expression on healthy activated T cells and B cells, CD30 expression has also been associated with some hematopoietic malignancies. See, e.g., van der Weyden et al. Nature Cancer Journal (2017) 7: e603. CD30 has also been reported to be expressed by hematopoietic stem and progenitor cells (HSPCs) during activation. See, e.g., Grover et al. BMC Cancer (2019) 19: 203. Approximately 20% of B-cell lymphoid malignancies and approximately 30% of T-cell lymphoid malignancies have been found to express CD30. See, e.g., Sauder et al. Hematol. Oncol. Clin. North Am. (2017) 31(2): 317-334.

For example, there have been a number of clinical trials targeting CD30, for example using anti-CD30 antibodies, anti-CD30 antibody drug conjugates (e.g., brentuximab vedotin), and anti-CD30 chimeric antigen receptor (CAR) T cells, for the treatment of Hodgkin lymphoma and systemic anaplastic large-cell lymphoma, and other CD30-expressing lymphomas.

Due to the shared expression of CD30 on both normal, activated healthy cells as well as being an expressed antigen on malignant cells, therapeutic targeting of CD30 may result in suppression of immune responses, such as those involving activated T cells and B cells.

Described herein are gRNAs that have been developed to specifically direct genetic modification of the gene encoding CD30. Also provided herein is use of such gRNAs to produce genetically modified cells, such as hematopoietic cells, immune cells, lymphocytes, and populations of such cells, that are deficient in CD30 or have reduced expression of CD30 such that the modified cells are not recognized by CD30-specific immunotherapies. Also provided herein are methods involving administering such cells, or compositions thereof, to subjects to address the problem of on-target, off-disease cytotoxicity of certain immunotherapeutic agents. In some examples, as described herein, the genetically modified cells are hematopoietic cells that are deficient in CD30 or have reduced expression of CD30 that are capable, for example, of developing into lineage-committed cells, such as T cells that are deficient in CD30 or have reduced expression of CD30, and therefore, are resistant to killing by CD30-specific immunotherapies. Alternatively or in addition, in some examples, as described herein, the genetically modified cells are immune cells, such as CD30-specific CAR T cells that are deficient in in CD30 or have reduced expression of CD30, and therefore, are resistant to fratricide killing by other CD30-specific CAR T cells.

### Genetically Engineered Cells and Related Compositions and Methods

Some aspects of this disclosure provide genetically engineered cells comprising a modification in their genome that results in a loss of expression of CD30, or expression of a variant form of CD30 that is not recognized by an immunotherapeutic agent targeting CD30. In some embodiments, the modification in the genome of the cell is a mutation in a genomic sequence encoding CD30.

The term “mutation,” as used herein, refers to a change (e.g., an insertion, deletion, inversion, or substitution) in a nucleic acid sequence as compared to a reference sequence, e.g., the corresponding sequence of a cell not having such a mutation, or the corresponding wild-type nucleic acid sequence. In some embodiments provided herein, a mutation in a gene encoding CD30 results in a loss of expression of CD30 in a cell harboring the mutation. In some embodiments, a mutation in a gene encoding CD30 results in the expression of a variant form of CD30 that is not bound by an immunotherapeutic agent targeting CD30, or bound at a significantly lower level than the non-mutated CD30 form encoded by the gene. In some embodiment, a cell harboring a genomic mutation in the CD30 gene as provided herein is not bound by, or is bound at a significantly lower level by an immunotherapeutic agent that targets CD30, e.g., an anti-CD30 antibody or chimeric antigen receptor (CAR).

Some aspects of this disclosure provide compositions and methods for generating the genetically engineered cells described herein, e.g., genetically engineered cells comprising a modification in their genome that results in a loss of expression of CD30, or expression of a variant form of CD30 that is not recognized by an immunotherapeutic agent targeting CD30. Such compositions and methods provided herein include, without limitation, suitable strategies and approaches for genetically engineering cells, e.g., by using RNA-guided nucleases, such as CRISPR/Cas nucleases, and suitable RNAs able to bind such RNA-guided nucleases and target them to a suitable target site within the genome of a cell to effect a genomic modification resulting in a loss of expression of CD30, or expression of a variant form of CD30 that is not recognized by an immunotherapeutic agent targeting CD30.

In some embodiments, a genetically engineered cell (e.g., a genetically engineered hematopoietic cell, such as, for example, a genetically engineered hematopoietic stem or progenitor cell or a genetically engineered immune effector cell) described herein is generated via genome editing technology, which includes any technology capable of introducing targeted changes, also referred to as “edits,” into the genome of a cell.

One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. See, Yeh et al. Nat. Cell. Biol. (2019) 21: 1468-1478; e.g., Hsu et al. Cell (2014) 157: 1262-1278; Jasin et al. DNA Repair (2016) 44: 6-16; Sfeir et al. Trends Biochem. Sci. (2015) 40: 701-714.

Another exemplary suitable genome editing technology is “base editing,” which includes the use of a base editor, e.g., a nuclease-impaired or partially nuclease-impaired RNA-guided CRISPR/Cas protein fused to a deaminase that targets and deaminates a specific nucleobase, e.g., a cytosine or adenosine nucleobase of a C or A nucleotide, which, via cellular mismatch repair mechanisms, results in a change from a C to a T nucleotide, or a change from an A to a G nucleotide. See, e.g., Komor et al. Nature (2016) 533: 420-424; Rees et al. Nat. Rev. Genet. (2018) 19(12): 770-788; Anzaolne et al. Nat. Biotechnol. (2020) 38: 824-844;

Yet another exemplary suitable genome editing technology includes “prime editing,” which includes the introduction of new genetic information, e.g., an altered nucleotide sequence, into a specifically targeted genomic site using a catalytically impaired or partially catalytically impaired RNA-guided nuclease, e.g., a CRISPR/Cas nuclease, fused to an engineered reverse transcriptase (RT) domain. The Cas/RT fusion is targeted to a target site within the genome by a guide RNA that also comprises a nucleic acid sequence encoding the desired edit, and that can serve as a primer for the RT. See, e.g., Anzalone et al. Nature (2019) 576 (7785): 149-157.

The use of genome editing technology typically features the use of a suitable RNA-guided nuclease, which, in some embodiments, e.g., for base editing or prime editing, may be catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., an spCas9 or an saCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas12a orthologs, and Cas12a derivatives, such as the MAD7 system (MAD7™, Inscripta, Inc.), or the Alt-R Cas12a (Cpfl) Ultra nuclease (Alt-R® Cas12a Ultra; Integrated DNA Technologies, Inc.). See, e.g., Gill et al. LIPSCOMB 2017. In United States: Inscripta Inc.; Price et al. Biotechnol. Bioeng. (2020) 117(60): 1805-1816;

In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs are described in more detail elsewhere herein.

In some embodiments, a CD30 gRNA described herein is complexed with a CRISPR/Cas nuclease, e.g., a Cas9 nuclease. Various Cas9 nucleases are suitable for use with the gRNAs provided herein to effect genome editing according to aspects of this disclosure, e.g., to create a genomic modification in the CD30 gene. Typically, the Cas nuclease and the gRNA are provided in a form and under conditions suitable for the formation of a Cas/gRNA complex, that targets a target site on the genome of the cell, e.g., a target site within the CD30 gene. In some embodiments, a Cas nuclease is used that exhibits a desired PAM specificity to target the Cas/gRNA complex to a desired target domain in the CD30 gene. Suitable target domains and corresponding gRNA targeting domain sequences are provided herein.

In some embodiments, a Cas/gRNA complex is formed, e.g., in vitro, and a target cell is contacted with the Cas/gRNA complex, e.g., via electroporation of the Cas/gRNA complex into the cell. In some embodiments, the cell is contacted with Cas protein and gRNA separately, and the Cas/gRNA complex is formed within the cell. In some embodiments, the cell is contacted with a nucleic acid, e.g., a DNA or RNA, encoding the Cas protein, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In some embodiments, catalytically impaired, or partially impaired, variants of such Cas9 nucleases may be used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type V-B, type V-C, and type V-U. See, e.g., Stella et al. Nature Structural & Molecular Biology (2017). In some embodiments, the Cas nuclease is a type V-B Cas endonuclease, such as a C2c1. See, e.g., Shmakov et al. Mol Cell (2015) 60: 385-397. In some embodiments, the Cas nuclease used in the methods of genome editing provided herein is a type V-A Cas endonuclease, such as a Cpfl (Cas12a) nuclease. See, e.g., Strohkendl et al. Mol. Cell (2018) 71: 1-9. In some embodiments, a Cas nuclease used in the methods of genome editing provided herein is a Cpfl nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7™ (Inscripta).

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1domain is involved in recognition of the repeat:anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1domain. The REC2 domain, or parts thereof, may also play a role in the recognition of the repeat: anti-repeat duplex. The REC2 domain comprises amino acids 180-307 of the sequence of S. pyogenes Cas9.

The NUC lobe comprises the RuvC domain (also referred to herein as RuvC-like domain), the HNH domain (also referred to herein as HNH-like domain), and the PAM-interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343(6176): 1247997, 2014) and for S. pyogenes Cas9 with a guide RNA (e.g., a synthetic fusion of crRNA and tracrRNA) (Nishimasu et al., Cell (2014) 156:935-949; and Anders et al., Nature (2014) doi: 10.1038/naturel3579).

In some embodiments, a Cas9 molecule described herein exhibits nuclease activity that results in the introduction of a double strand DNA break in or directly proximal to a target site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage). In some embodiments, the Cas9 molecule is an enhanced specificity Cas9 variant (e.g., eSPCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529: 490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, spCas9 and saCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168: 20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpfl endonuclease comprising substitution mutations S542R, K607R, and N552R recognizes the PAM sequence TATV. See, e.g., Gao et al. Nat. Biotechnol. (2017) 35(8): 789-792.

In some embodiments, more than one (e.g., 2, 3, or more) Cas9 molecules are used. In some embodiments, at least one of the Cas9 molecule is a Cas9 enzyme. In some embodiments, at least one of the Cas molecules is a Cpfl enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes.

In some embodiments, a base editor is used to create a genomic modification resulting in a loss of expression of CD30, or in expression of a CD30 variant not targeted by an immunotherapy. Base editors typically comprise a catalytically inactive or partially inactive Cas nuclease fused to a functional domain, e.g., a deaminase domain. See, e.g., Eid et al. Biochem. J. (2018) 475(11): 1955-1964; Rees et al. Nature Reviews Genetics (2018) 19:770-788. In some embodiments, a catalytically inactive Cas nuclease is referred to as “dead Cas” or “dCas.” In some embodiments, the endonuclease comprises a dCas fused to an adenine base editor (ABE), for example an ABE evolved from the RNA adenine deaminase TadA. In some embodiments, the endonuclease comprises a dCas fused to cytidine deaminase enzyme (e.g., APOBEC deaminase, pmCDA1, activation-induced cytidine deaminase (AID)). In some embodiments, the catalytically inactive Cas molecule has reduced activity and is, e.g., a nickase (referred to as “nCas”).

In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to cytidine deaminase enzyme (e.g., APOBEC deaminase, pmCDA1, activation-induced cytidine deaminase (AID)). In some embodiments, the catalytically inactive Cas9 molecule has reduced activity and is nCas9. In some embodiments, the catalytically inactive Cas9 molecule (dCas9) is fused to one or more uracil glycosylase inhibitor (UGI) domains. In some embodiments, the Cas9 molecule comprises an inactive Cas9 molecule (dCas9) fused to an adenine base editor (ABE), for example an ABE evolved from the RNA adenine deaminase TadA. In some embodiments, the Cas9 molecule comprises a nCas9 fused to an adenine base editor (ABE), for example an ABE evolved from the RNA adenine deaminase TadA. In some embodiments, the Cas9 molecule comprises a dCas9 fused to cytidine deaminase enzyme (e.g., APOBEC deaminase, pmCDA1, activation-induced cytidine deaminase (AID)). In some embodiments, the Cas9 molecule comprises a nCas9 fused to cytidine deaminase enzyme (e.g., APOBEC deaminase, pmCDA1, activation-induced cytidine deaminase (AID)).

Examples of suitable base editors include, without limitation, BE1, BE2, BE3, HF-BE3, BE4, BE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, VQR-ABE, VRER-ABE, Sa(KKH)-ABE, and CRISPR-SKIP. Additional examples of base editors can be found, for example, in U.S. Publication No. 2018/0312825A1, U.S. Publication No. 2018/0312828A1, and PCT Publication No. WO 2018/165629A1, which are incorporated by reference herein in their entireties.

Some aspects of this disclosure provide guide RNAs that are suitable to target an RNA-guided nuclease, e.g. as provided herein, to a suitable target site in the genome of a cell in order to effect a modification in the genome of the cell that results in a loss of expression of CD30, or expression of a variant form of CD30 that is not recognized by an immunotherapeutic agent targeting CD30.

The terms “guide RNA” and “gRNA” are used interchangeably herein and refer to a nucleic acid, typically an RNA, that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA-guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNA sequence).

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337(6096):816-821, Ran, et al. Nature Protocols (2013) 8:2281-2308, PCT Publication No. WO2014/093694, and PCT Publication No. WO2013/176772.

For example, the binding domains of naturally occurring spCas9 gRNA typically comprise two RNA molecules, the crRNA (partially) and the tracrRNA. Variants of spCas9 gRNAs that comprise only a single RNA molecule including both crRNA and tracrRNA sequences, covalently bound to each other, e.g., via a tetraloop or via click-chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

A gRNA suitable for targeting a target site in the CD30 gene may comprise a number of domains. In some embodiments, e.g., in some embodiments where a Cas9 nuclease is used, a unimolecular sgRNA, may comprise, from 5′ to 3′:
a targeting domain corresponding to a target site sequence in the CD30 gene;
a first complementarity domain;
a linking domain;
a second complementarity domain (which is complementary to the first complementarity domain);
a proximal domain; and
optionally, a tail domain.

Each of these domains is now described in more detail.

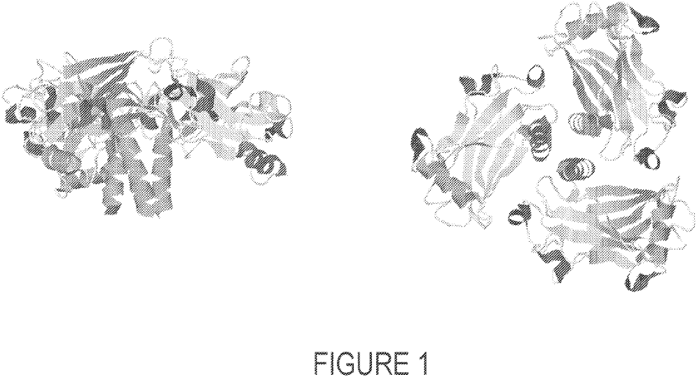

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., FIG. 1 of Vanegas et al., Fungal Biol Biotechnol. 2019; 6: 6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg SH et al., Nature 2014 (doi: 10.1038/naturel3011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

[ target domain (DNA) ] [ PAM ]
5'-N-N-N-N-N-N-N-N-N-N-N-N-N-N-N-N-N-N-N-N-N-N-N-G-G-3' (DNA)
3'-N-N-N-N-N-N-N-N-N-N-N-N-N-N-N-N-N-N-N-N-N-N-N-C-C-5' (DNA)
| | | | | | | | | | | | | | | | | | | | | |
5'-N-N-N-N-N-N-N-N-N-N-N-N-N-N-N-N-N-N-N-N-N-N-[gRNA scaffold]-3' (RNA)
[ targeting domain (RNA) ][binding domain]

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

[ PAM ] [ target domain (DNA) ]
5'-T-T-N-N-N-N-N-N-N-N-N-N-N-N-N-N-N-N-N-N-N-N-N-N-N-3' (DNA)
3'-A-A-N-N-N-N-N-N-N-N-N-N-N-N-N-N-N-N-N-N-N-N-N-N-N-5' (DNA)
| | | | | | | | | | | | | | | | | | | | | |
5'-[gRNA scaffold]-N-N-N-N-N-N-N-N-N-N-N-N-N-N-N-N-N-N-N-N-N-N-3' (RNA)
[binding domain][ targeting domain (RNA) ]

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24 or 25 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises:
a first strand comprising, e.g., from 5′ to 3′:
   a targeting domain (which corresponds to a target domain in the CD30 gene); and
   a first complementarity domain; and
a second strand, comprising, e.g., from 5′ to 3′:
   optionally, a 5′ extension domain;
   a second complementarity domain;
   a proximal domain; and
   optionally, a tail domain.

In some embodiments, any of the gRNAs provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33(9): 985-989, each of which is incorporated herein by reference in its entirety.

For example, a gRNA provided herein may comprise one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-O-methyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, a gRNA provided herein may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g. 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-O-methyl-3′-phosphorothioate nucleotides, e.g., at least 1, 2, 3, 4, 5, or 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA may comprise one or more modified nucleotides, e.g., as described in PCT Publication Nos. WO2017/214460, WO2016/089433, and WO2016/164356, which are incorporated by reference their entirety.

The CD30 targeting gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

The present disclosure provides a number of CD30 target sites and corresponding gRNAs that are useful for targeting an RNA-guided nuclease to human CD30. Table 1 below illustrates preferred target domains in the human endogenous CD30 gene that can be bound by gRNAs described herein. The exemplary target sequences of human CD30 shown in Table 1, in some embodiments, are for use with a Cas9 nuclease, e.g., SpCas9.

TABLE 1

Exemplary Cas9 target site sequences of human CD30 are provided, as are exemplary gRNA targeting domain sequences useful for targeting such sites

| | |
|---|---|
| gRNA Name | Target domain sequence |
| CD30-1 | ACATGCCGGGTCGACATTCG (SEQ ID NO: 1) |
| | CGAATGTCGACCCGGCATGT (SEQ ID NO: 28) |
| | ACAUGCCGGGUCGACAUUCG (SEQ ID NO: 55) |
| CD30-2 | CGTGTCTGCGAATGTCGACC (SEQ ID NO: 2) |
| | GGTCGACATTCGCAGACACG (SEQ ID NO: 29) |
| | CGUGUCUGCGAAUGUCGACC (SEQ ID NO: 56) |
| CD30-3 | TCGACATTCGCAGACACGGG (SEQ ID NO: 3) |
| | CCCGTGTCTGCGAATGTCGA (SEQ ID NO: 30) |
| | UCGACAUUCGCAGACACGGG (SEQ ID NO: 57) |
| CD30-4 | GTGTCCCTTAGACGACCTCG (SEQ ID NO: 4) |
| | CGAGGTCGTCTAAGGGACAC (SEQ ID NO: 31) |
| | GUGUCCCUUAGACGACCUCG (SEQ ID NO: 58) |
| CD30-5 | GTCCCTTAGACGACCTCGTG (SEQ ID NO: 5) |
| | CACGAGGTCGTCTAAGGGAC (SEQ ID NO: 32) |
| | GUCCCUUAGACGACCUCGUG (SEQ ID NO: 59) |
| CD30-6 | GTCGACATTCGCAGGTGCGG (SEQ ID NO: 6) |
| | CCGCACCTGCGAATGTCGAC (SEQ ID NO: 33) |
| | GUCGACAUUCGCAGGUGCGG (SEQ ID NO: 60) |
| CD30-7 | CGAGGCCCTTACCTCGAGAA (SEQ ID NO: 7) |
| | TTCTCGAGGTAAGGGCCTCG (SEQ ID NO: 34) |
| | CGAGGCCCUUACCUCGAGAA (SEQ ID NO: 61) |
| CD30-8 | CTGCCAGGATCGACCCTTCG (SEQ ID NO: 8) |
| | CGAAGGGTCGATCCTGGCAG (SEQ ID NO: 35) |
| | CUGCCAGGAUCGACCCUUCG (SEQ ID NO: 62) |
| CD30-9 | ACCTCGAGAACAAGTCACGC (SEQ ID NO: 9) |
| | GCGTGACTTGTTCTCGAGGT (SEQ ID NO: 36) |
| | ACCUCGAGAACAAGUCACGC (SEQ ID NO: 63) |
| CD30-10 | CGTGACTTGTTCTCGAGGTA (SEQ ID NO: 10) |
| | TACCTCGAGAACAAGTCACG (SEQ ID NO: 37) |
| | CGUGACUUGUUCUCGAGGUA (SEQ ID NO: 64) |
| CD30-11 | CATGCCAGGTCGACATTCGC (SEQ ID NO: 11) |
| | GCGAATGTCGACCTGGCATG (SEQ ID NO: 38) |
| | CAUGCCAGGUCGACAUUCGC (SEQ ID NO: 65) |
| CD30-12 | GGGAGCCCTCGTCAGTTTAG (SEQ ID NO: 12) |
| | CTAAACTGACGAGGGCTCCC (SEQ ID NO: 39) |
| | GGGAGCCCUCGUCAGUUUAG (SEQ ID NO: 66) |
| CD30-13 | CGTGAGCTGTTCTCGAGGTA (SEQ ID NO: 13) |
| | TACCTCGAGAACAGCTCACG (SEQ ID NO: 40) |
| | CGUGAGCUGUUCUCGAGGUA (SEQ ID NO: 67) |
| CD30-14 | ACCTCGAGAACAGCTCACGC (SEQ ID NO: 14) |
| | GCGTGAGCTGTTCTCGAGGT (SEQ ID NO: 41) |
| | ACCUCGAGAACAGCUCACGC (SEQ ID NO: 68) |
| CD30-15 | GCGTGACTTGTTCTCGAGGT (SEQ ID NO: 15) |
| | ACCTCGAGAACAAGTCACGC (SEQ ID NO: 42) |
| | GCGUGACUUGUUCUCGAGGU (SEQ ID NO: 69) |
| CD30-16 | AGCCCGACTACTACCTGGAC (SEQ ID NO: 16) |
| | GTCCAGGTAGTAGTCGGGCT (SEQ ID NO: 43) |
| | AGCCCGACUACUACCUGGAC (SEQ ID NO: 70) |
| CD30-17 | GCCCGTCACCCGCTTACCTG (SEQ ID NO: 17) |
| | CAGGTAAGCGGGTGACGGGC (SEQ ID NO: 44) |
| | GCCCGUCACCCGCUUACCUG (SEQ ID NO: 71) |
| CD30-18 | TTCTCCACGAGGTCGTCTAA (SEQ ID NO: 18) |
| | TTAGACGACCTCGTGGAGAA (SEQ ID NO: 45) |
| | UUCUCCACGAGGUCGUCUAA (SEQ ID NO: 72) |
| CD30-19 | GAGGAGGACGCGCATCCCCG (SEQ ID NO: 19) |
| | CGGGGATGCGCGTCCTCCTC (SEQ ID NO: 46) |
| | GAGGAGGACGCGCAUCCCCG (SEQ ID NO: 73) |
| CD30-20 | GCCTGCGTGACTTGTTCTCG (SEQ ID NO: 20) |
| | CGAGAACAAGTCACGCAGGC (SEQ ID NO: 47) |
| | GCCUGCGUGACUUGUUCUCG (SEQ ID NO: 74) |
| CD30-21 | CACACGGCGTCTTCTCCACG (SEQ ID NO: 21) |
| | CGTGGAGAAGACGCCGTGTG (SEQ ID NO: 48) |
| | CACACGGCGUCUUCUCCACG (SEQ ID NO: 75) |
| CD30-22 | AAGGCCCTTACCTCGAGAAC (SEQ ID NO: 22) |
| | GTTCTCGAGGTAAGGGCCTT (SEQ ID NO: 49) |
| | AAGGCCCUUACCUCGAGAAC (SEQ ID NO: 76) |
| CD30-23 | AAGGGTCGATCCTGGCAGGT (SEQ ID NO: 23) |
| | ACCTGCCAGGATCGACCCTT (SEQ ID NO: 50) |
| | AAGGGUCGAUCCUGGCAGGU (SEQ ID NO: 77) |
| CD30-24 | CAGGTCGACATTCGCAGGTG (SEQ ID NO: 24) |
| | CACCTGCGAATGTCGACCTG (SEQ ID NO: 51) |
| | CAGGUCGACAUUCGCAGGUG (SEQ ID NO: 78) |
| CD30-25 | AATCATCCCTGCCGGACAGA (SEQ ID NO: 25) |
| | TCTGTCCGGCAGGGATGATT (SEQ ID NO: 52) |
| | AAUCAUCCCUGCCGGACAGA (SEQ ID NO: 79) |
| CD30-26 | AACAGCTCACGCAGGCCGTG (SEQ ID NO: 26) |
| | CACGGCCTGCGTGAGCTGTT (SEQ ID NO: 53) |
| | AACAGCUCACGCAGGCCGUG (SEQ ID NO: 80) |
| CD30-27 | GCAGTCCCAGCGCGGCGAGG (SEQ ID NO: 27) |
| | CCTCGCCGCGCTGGGACTGC (SEQ ID NO: 54) |
| | GCAGUCCCAGCGCGGCGAGG (SEQ ID NO: 81) |

For each target site, the first sequence represents the DNA target domain sequence, the second sequence represents the reverse complement thereof, and the third sequence represents an exemplary targeting domain sequence of a gRNA that can be used to target the respective target site.

The present disclosure provides exemplary CD30 targeting gRNAs that are useful for targeting an RNA-guided nuclease to human CD30. Table 2 below illustrates preferred targeting domains for use in gRNAs targeting Cas9 nucleases to human endogenous CD30 gene. The exemplary target sequences of human CD30 shown in Table 2, in some embodiments, are for use with a Cas9 nuclease, e.g., SpCas9.

TABLE 2

Exemplary Cas9 targeting domain sequences of gRNAs targeted to human CD30 are provided

| | |
|---|---|
| gRNA Name | Targeting domain sequence |
| CD30-1 | ACAUGCCGGGUCGACAUUCG (SEQ ID NO: 55) |
| CD30-2 | CGUGUCUGCGAAUGUCGACC (SEQ ID NO: 56) |
| CD30-3 | UCGACAUUCGCAGACACGGG (SEQ ID NO: 57) |
| CD30-4 | GUGUCCCUUAGACGACCUCG (SEQ ID NO: 58) |
| CD30-5 | GUCCCUUAGACGACCUCGUG (SEQ ID NO: 59) |
| CD30-6 | GUCGACAUUCGCAGGUGCGG (SEQ ID NO: 60) |
| CD30-7 | CGAGGCCCUUACCUCGAGAA (SEQ ID NO: 61) |
| CD30-8 | CUGCCAGGAUCGACCCUUCG (SEQ ID NO: 62) |
| CD30-9 | ACCUCGAGAACAAGUCACGC (SEQ ID NO: 63) |
| CD30-10 | CGUGACUUGUUCUCGAGGUA (SEQ ID NO: 64) |
| CD30-11 | CAUGCCAGGUCGACAUUCGC (SEQ ID NO: 65) |
| CD30-12 | GGGAGCCCUCGUCAGUUUAG (SEQ ID NO: 66) |
| CD30-13 | CGUGAGCUGUUCUCGAGGUA (SEQ ID NO: 67) |
| CD30-14 | ACCUCGAGAACAGCUCACGC (SEQ ID NO: 68) |
| CD30-15 | GCGUGACUUGUUCUCGAGGU (SEQ ID NO: 69) |
| CD30-16 | AGCCCGACUACUACCUGGAC (SEQ ID NO: 70) |
| CD30-17 | GCCCGUCACCCGCUUACCUG (SEQ ID NO: 71) |
| CD30-18 | UUCUCCACGAGGUCGUCUAA (SEQ ID NO: 72) |
| CD30-19 | GAGGAGGACGCGCAUCCCCG (SEQ ID NO: 73) |
| CD30-20 | GCCUGCGUGACUUGUUCUCG (SEQ ID NO: 74) |
| CD30-21 | CACACGGCGUCUUCUCCACG (SEQ ID NO: 75) |
| CD30-22 | AAGGCCCUUACCUCGAGAAC (SEQ ID NO: 76) |
| CD30-23 | AAGGGUCGAUCCUGGCAGGU (SEQ ID NO: 77) |
| CD30-24 | CAGGUCGACAUUCGCAGGUG (SEQ ID NO: 78) |
| CD30-25 | AAUCAUCCCUGCCGGACAGA (SEQ ID NO: 79) |
| CD30-26 | AACAGCUCACGCAGGCCGUG (SEQ ID NO: 80) |
| CD30-27 | GCAGUCCCAGCGCGGCGAGG (SEQ ID NO: 81) |

The present disclosure provides a number of CD30 target sites and corresponding gRNAs that are useful for targeting an RNA-guided nuclease to human CD30. Table 3 below illustrates preferred target domains in the human endogenous CD30 gene that can be bound by gRNAs described herein. The exemplary target sequences of human CD30 shown in Table 3, in some embodiments, are for use with a Cpfl nuclease.

TABLE 3

Exemplary Cas12a/Cpf1 target site sequences of human CD30 are provided, as are exemplary gRNA targeting domain sequences useful for targeting such sites

| gRNA Name | Target Domain Sequences |
|---|---|
| CD30-28 | GACGGCTGGACACTCACCCAC (SEQ ID NO: 82) |
| | GTGGGTGAGTGTCCAGCCGTC (SEQ ID NO: 92) |
| | GACGGCUGGACACUCACCCAC (SEQ ID NO: 102) |
| CD30-29 | TGACCTGCCAGGATCGACCCT (SEQ ID NO: 83) |
| | AGGGTCGATCCTGGCAGGTCA (SEQ ID NO: 93) |
| | UGACCUGCCAGGAUCGACCCU (SEQ ID NO: 103) |
| CD30-30 | CATGACAGGTGTCCTCGAAGG (SEQ ID NO: 84) |
| | CCTTCGAGGACACCTGTCATG (SEQ ID NO: 94) |
| | CAUGACAGGUGUCCUCGAAGG (SEQ ID NO: 104) |
| CD30-31 | AGGCGCCACCCCTGGGGACCC (SEQ ID NO: 85) |
| | GGGTCCCCAGGGGTGGCGCCT (SEQ ID NO: 95) |
| | AGGCGCCACCCCUGGGGACCC (SEQ ID NO: 105) |
| CD30-32 | CCAGGGCTGTTCCCGACACAG (SEQ ID NO: 86) |
| | CTGTGTCGGGAACAGCCCTGG (SEQ ID NO: 96) |
| | CCAGGGCUGUUCCCGACACAG (SEQ ID NO: 106) |
| CD30-33 | GAAGCAGCTTCCTGGGCGAGG (SEQ ID NO: 87) |
| | CCTCGCCCAGGAAGCTGCTTC (SEQ ID NO: 97) |
| | GAAGCAGCUUCCUGGGCGAGG (SEQ ID NO: 107) |
| CD30-34 | AGAGCTCCACCTGTGCTACCC (SEQ ID NO: 88) |
| | GGGTAGCACAGGTGGAGCTCT (SEQ ID NO: 98) |
| | AGAGCUCCACCUGUGCUACCC (SEQ ID NO: 108) |
| CD30-35 | CCCAGGGCTGTTCCCGACACA (SEQ ID NO: 89) |
| | TGTGTCGGGAACAGCCCTGGG (SEQ ID NO: 99) |
| | CCCAGGGCUGUUCCCGACACA (SEQ ID NO: 109) |
| CD30-36 | GCAGATTCCAGACCCAGGAGG (SEQ ID NO: 90) |
| | CCTCCTGGGTCTGGAATCTGC (SEQ ID NO: 100) |
| | GCAGAUUCCAGACCCAGGAGG (SEQ ID NO: 110) |
| CD30-37 | TGCAATCACCAGACCCCTCTG (SEQ ID NO: 91) |
| | CAGAGGGGTCTGGTGATTGCA (SEQ ID NO: 101) |
| | UGCAAUCACCAGACCCCUCUG (SEQ ID NO: 111) |

For each target site, the first sequence represents the DNA target domain sequence, the second sequence represents the reverse complement thereof, and the third sequence represents an exemplary targeting domain sequence of a gRNA that can be used to target the respective target site.

The present disclosure provides exemplary CD30 targeting gRNAs that are useful for targeting an RNA-guided nuclease to human CD30. Table 4 below illustrates preferred targeting domains for use in gRNAs targeting a RNA-guided nuclease to human endogenous CD30 gene. The exemplary target sequences of human CD30 shown in Table 4, in some embodiments, are for use with a Cpfl nuclease.

TABLE 4

Exemplary Cas12a/Cpf1 targeting domain sequences of gRNAs targeted to human CD30 are provided

| | |
|---|---|
| gRNA Name | Target Domain Sequences |
| CD30-28 | GACGGCUGGACACUCACCCAC (SEQ ID NO: 102) |
| CD30-29 | UGACCUGCCAGGAUCGACCCU (SEQ ID NO: 103) |
| CD30-30 | CAUGACAGGUGUCCUCGAAGG (SEQ ID NO: 104) |
| CD30-31 | AGGCGCCACCCCUGGGGACCC (SEQ ID NO: 105) |
| CD30-32 | CCAGGGCUGUUCCCGACACAG (SEQ ID NO: 106) |
| CD30-33 | GAAGCAGCUUCCUGGGCGAGG (SEQ ID NO: 107) |
| CD30-34 | AGAGCUCCACCUGUGCUACCC (SEQ ID NO: 108) |
| CD30-35 | CCCAGGGCUGUUCCCGACACA (SEQ ID NO: 109) |
| CD30-36 | GCAGAUUCCAGACCCAGGAGG (SEQ ID NO: 110) |
| CD30-37 | UGCAAUCACCAGACCCCUCUG (SEQ ID NO: 111) |

A representative amino acid sequence of CD30 is provided by UniProtKB/Swiss-Prot Accession No. P28908, shown below.

MRVLLAALGLLFLGALRAFPQDRPFEDTCHGNPSHYYDKAVRRCCYRCPM
GLFPTQQCPQRPTDCRKQCEPDYYLDEADRCTACVTCSRDDLVEKTPCAW
NSSRVCECRPGMFCSTSAVNSCARCFFHSVCPAGMIVKFPGTAQKNTVCE
PASPGVSPACASPENCKEPSSGTIPQAKPTPVSPATSSASTMPVRGGTRL
AQEAASKLTRAPDSPSSVGRPSSDPGLSPTQPCPEGSGDCRKQCEPDYYL
DEAGRCTACVSCSRDDLVEKTPCAWNSSRTCECRPGMICATSATNSCARC
VPYPICAAETVTKPQDMAEKDTTFEAPPLGTQPDCNPTPENGEAPASTSP
TQSLLVDSQASKTLPIPTSAPVALSSTGKPVLDAGPVLFWVILVLVVVVG
SSAFLLCHRRACRKRIRQKLHLCYPVQTSQPKLELVDSRPRRSSTQLRSG
ASVTEPVAEERGLMSQPLMETCHSVGAAYLESLPLQDASPAGGPSSPRDL
PEPRVSTEHTNNKIEKIYIMKADTVIVGTVKAELPEGRGLAGPAEPELEE
ELEADHTPHYPEQETEPPLGSCSDVMLSVEEEGKEDPLPTAASGK (SEQ
ID NO: 112)

A representative DNA sequence of CD30 is provided by NCBI Reference Sequence No. NM_001243.5, shown below.

CTGAGTCATCTCTGCACGTGTTTGCCCCCTTTTTTCTTCGCTGCTTGTAG
CTAAGTGTTCCTGGAACCAATTTGATACGGGAGAACTAAGGCTGAAACCT
CGGAGGAACAACCACTTTTGAAGTGACTTCGCGGCGTGCGTTGGGTGCGG
ACTAGGTGGCCGCGGCGGGAGTGTGCTGGAGCCTGAAGTCCACGCGCGCG
GCTGAGAACCGCCGGGACCGCACGTGGGCGCCGCGCGCTTCCCCCGCTTC
CCAGGTGGGCGCCGGCCGCCAGGCCACCTCACGTCCGGCCCCGGGGATGC
GCGTCCTCCTCGCCGCGCTGGGACTGCTGTTCCTGGGGGCGCTACGAGCC
TTCCCACAGGATCGACCCTTCGAGGACACCTGTCATGGAAACCCCAGCCA
CTACTATGACAAGGCTGTCAGGAGGTGCTGTTACCGCTGCCCCATGGGGC
TGTTCCCGACACAGCAGTGCCCACAGAGGCCTACTGACTGCAGGAAGCAG
TGTGAGCCTGACTACTACCTGGATGAGGCCGACCGCTGTACAGCCTGCGT
GACTTGTTCTCGAGACGACCTCGTGGAGAAGACGCCGTGTGCATGGAACT
CCTCCCGTGTCTGCGAATGTCGACCCGGCATGTTCTGTTCCACGTCTGCC
GTCAACTCCTGTGCCCGCTGCTTCTTCCATTCTGTCTGTCCGGCAGGGAT
GATTGTCAAGTTCCCAGGCACGGCGCAGAAGAACACGGTCTGTGAGCCGG
CTTCCCCAGGGGTCAGCCCTGCCTGTGCCAGCCCAGAGAACTGCAAGGAA
CCCTCCAGTGGCACCATCCCCCAGGCCAAGCCCACCCCGGTGTCCCCAGC
AACCTCCAGTGCCAGCACCATGCCTGTAAGAGGGGGCACCCGCCTCGCCC
AGGAAGCTGCTTCTAAACTGACGAGGGCTCCCGACTCTCCCTCCTCTGTG
GGAAGGCCTAGTTCAGATCCAGGTCTGTCCCCAACACAGCCATGCCCAGA
GGGGTCTGGTGATTGCAGAAAGCAGTGTGAGCCCGACTACTACCTGGACG
AGGCCGGCCGCTGCACGGCCTGCGTGAGCTGTTCTCGAGATGACCTTGTG
GAGAAGACGCCATGTGCATGGAACTCCTCCCGCACCTGCGAATGTCGACC
TGGCATGATCTGTGCCACATCAGCCACCAACTCCTGTGCCCGCTGTGTCC
CCTACCCAATCTGTGCAGCAGAGACGGTCACCAAGCCCCAGGATATGGCT
GAGAAGGACACCACCTTTGAGGCGCCACCCCTGGGGACCCAGCCGGACTG
CAACCCCACCCCAGAGAATGGCGAGGCGCCTGCCAGCACCAGCCCCACTC
AGAGCTTGCTGGTGGACTCCCAGGCCAGTAAGACGCTGCCCATCCCAACC
AGCGCTCCCGTCGCTCTCTCCTCCACGGGGAAGCCCGTTCTGGATGCAGG
GCCAGTGCTCTTCTGGGTGATCCTGGTGTTGGTTGTGGTGGTCGGCTCCA
GCGCCTTCCTCCTGTGCCACCGGAGGGCCTGCAGGAAGCGAATTCGGCAG
AAGCTCCACCTGTGCTACCCGGTCCAGACCTCCCAGCCCAAGCTAGAGCT
TGTGGATTCCAGACCCAGGAGGAGCTCAACGCAGCTGAGGAGTGGTGCGT
CGGTGACAGAACCCGTCGCGGAAGAGCGAGGGTTAATGAGCCAGCCACTG
ATGGAGACCTGCCACAGCGTGGGGGCAGCCTACCTGGAGAGCCTGCCGCT
GCAGGATGCCAGCCCGGCCGGGGGCCCCTCGTCCCCCAGGGACCTTCCTG
AGCCCCGGGTGTCCACGGAGCACACCAATAACAAGATTGAGAAAATCTAC
ATCATGAAGGCTGACACCGTGATCGTGGGGACCGTGAAGGCTGAGCTGCC
GGAGGGCCGGGGCCTGGCGGGGCCAGCAGAGCCCGAGTTGGAGGAGGAGC
TGGAGGCGGACCATACCCCCCACTACCCCGAGCAGGAGACAGAACCGCCT
CTGGGCAGCTGCAGCGATGTCATGCTCTCAGTGGAAGAGGAAGGGAAAGA
AGACCCCTTGCCCACAGCTGCCTCTGGAAAGTGAGGCCTGGGCTGGGCTG
GGGCTAGGAGGGCAGCAGGGTGGCCTCTGGGAGGCCAGGATGGCACTGTT
GGCACCGAGGTTGGGGGCAGAGGCCCATCTGGCCTGAACTGAGGCTCCAG
CATCTAGTGGTGGACCGGCCGGTCACTGCAGGGGTCTGGTGGTCTCTGCT
TGCATCCCCAACTTAGCTGTCCCCTGACCCAGAGCCTAGGGGATCCGGGG
CTTGTACAGAAGAGACAGTCCAAGGGGACTGGATCCCAGCAGTGATGTTG
GTTGAGGCAGCAAACAGATGGCAGGATGGGCACTGCCGAGAACAGCATTG
GTCCCAGAGCCCTGGGCATCAGACCTTAACCACCAGGCCCACAGCCCAGC
GAGGGAGAGGTCGTGAGGCCAGCTCCCGGGGCCCCTGTAACCCTACTCTC
CTCTCTCCCTGGACCTCAGAGGTGACACCCATTGGGCCCTTCCGGCATGC
CCCCAGTTACTGTAAATGTGGCCCCCAGTGGGCATGGAGCCAGTGCCTGT
GGTTGTTTCTCCAGAGTCAAAAGGGAAGTCGAGGGATGGGGCGTCGTCAG
CTGGCACTGTCTCTGCTGCAGCGGCCACACTGTACTCTGCACTGGTGTGA
GGGCCCCTGCCTGGACTGTGGGACCCTCCTGGTGCTGCCCACCTTCCCTG
TCCTGTAGCCCCCTCGGTGGGCCCAGGGCCTAGGGCCCAGGATCAAGTCA
CTCATCTCAGAATGTCCCCACCAATCCCCGCCACAGCAGGCGCCTCGGGT
CCCAGATGTCTGCAGCCCTCAGCAGCTGCAGACCGCCCCTCACCAACCCA
GAGAACCTGCTTTACTTTGCCCAGGGACTTCCTCCCCATGTGAACATGGG
GAACTTCGGGCCCTGCCTGGAGTCCTTGACCGCTCTCTGTGGGCCCCACC
CACTCTGTCCTGGGAAATGAAGAAGCATCTTCCTTAGGTCTGCCCTGCTT
GCAAATCCACTAGCACCGACCCCACCACCTGGTTCCGGCTCTGCACGCTT
TGGGGTGTGGATGTCGAGAGGCACCACGGCCTCACCCAGGCATCTGCTTT
ACTCTGGACCATAGGAAACAAGACCGTTTGGAGGTTTCATCAGGATTTTG
GGTTTTTCACATTTCACGCTAAGGAGTAGTGGCCCTGACTTCCGGTCGGC
TGGCCAGCTGACTCCCTAGGGCCTTCAGACGTGTATGCAAATGAGTGATG
GATAAGGATGAGTCTTGGAGTTGCGGGCAGCCTGGAGACTCGTGGACTTA
CCGCCTGGAGGCAGGCCCGGGAAGGCTGCTGTTTACTCATCGGGCAGCCA
CGTGCTCTCTGGAGGAAGTGATAGTTTCTGAAACCGCTCAGATGTTTTGG
GGAAAGTTGGAGAAGCCGTGGCCTTGCGAGAGGTGGTTACACCAGAACCT
GGACATTGGCCAGAAGAAGCTTAAGTGGGCAGACACTGTTTGCCCAGTGT
TTGTGCAAGGATGGAGTGGGTGTCTCTGCATCACCCACAGCCGCAGCTGT
AAGGCACGCTGGAAGGCACACGCCTGCCAGGCAGGGCAGTCTGGCGCCCA
TGATGGGAGGGATTGACATGTTTCAACAAAATAATGCACTTCCTTACCTA
GTGGCCCTTCACACAACTTTTGAATCTCTAAAAATCCATAAAATCCTTAA
AGAACTGTAA (SEQ ID NO:113)

A representative DNA sequence of CD30 is also provided by NCBI Reference Sequence No.: NG_029573.2.

Some aspects of this disclosure provide genetically engineered cells comprising a modification in their genome that results in a loss of expression of CD30, or expression of a variant form of CD30 that is not recognized by an immunotherapeutic agent targeting CD30. In some embodiments, the modification in the genome of the cell is a mutation in a genomic sequence encoding CD30. In some embodiments, the modification is effected via genome editing, e.g., using a Cas nuclease and a gRNA targeting a CD30 target site provided herein or comprising a targeting domain sequence provided herein.

While the compositions, methods, strategies, and treatment modalities provided herein may be applied to any cell or cell type, some exemplary cells and cell types that are particularly suitable for genomic modification in the CD30 gene according to aspects of this invention are described in more detail herein. The skilled artisan will understand, however, that the provision of such examples is for the purpose of illustrating some specific embodiments, and additional suitable cells and cell types will be apparent to the skilled artisan based on the present disclosure, which is not limited in this respect.

Some aspects of this disclosure provide genetically engineered hematopoietic cells comprising a modification in their genome that results in a loss of expression of CD30, or expression of a variant form of CD30 that is not recognized by an immunotherapeutic agent targeting CD30. In some embodiments, the genetically engineered cells comprising a modification in their genome results in reduced cell surface expression of CD30 and/or reduced binding by an immunotherapeutic agent targeting CD30, e.g., as compared to a hematopoietic cell of the same cell type but not comprising a genomic modification. In some embodiments, a hematopoietic cell is a hematopoietic stem cell (HSC). In some embodiments, the hematopoietic cell is a hematopoietic progenitor cell (HPC). In some embodiments, the hematopoietic cell is a hematopoietic stem or progenitor cell.

In some embodiments, the cells are CD34+. In some embodiments, the cell is a hematopoietic cell. In some embodiments, the cell is a hematopoietic stem cell. In some embodiments, the cell is a hematopoietic progenitor cell. In some embodiments, the cell is an immune effector cell. In some embodiments, the cell is a lymphocyte. In some embodiments, the cell is a T-lymphocyte. In some embodiments, the cell is a NK cell. In some embodiments, the cell is a stem cell. In some embodiments, the stem cell is selected from the group consisting of an embryonic stem cell (ESC), an induced pluripotent stem cell (iPSC), a mesenchymal stem cell, or a tissue-specific stem cell.

In some embodiments, the cells are comprised in a population of cells which is characterized by the ability to engraft CD30-edited hematopoietic stem cells in the bone marrow of a recipient and to generate differentiated progeny of all blood lineage cell types in the recipient. In some embodiments, the cell population is characterized by the ability to engraft CD30-edited hematopoietic stem cells in the bone marrow of a recipient at an efficiency of at least 50%. In some embodiments, the cell population is characterized by the ability to engraft CD30-edited hematopoietic stem cells in the bone marrow of a recipient at an efficiency of at least 60%. In some embodiments, the cell population is characterized by the ability to engraft CD30-edited hematopoietic stem cells in the bone marrow of a recipient at an efficiency of at least 70%. In some embodiments, the cell population is characterized by the ability to engraft CD30-edited hematopoietic stem cells in the bone marrow of a recipient at an efficiency of at least 80%. In some embodiments, the cell population is characterized by the ability to engraft CD30-edited hematopoietic stem cells in the bone marrow of a recipient at an efficiency of at least 90%. In some embodiments, the cell population comprises CD30-edited hematopoietic stem cells that are characterized by a differentiation potential that is equivalent to the differentiation potential of non-edited hematopoietic stem cells.

In some embodiments, a hematopoietic cell (e.g., an HSC or HPC) comprising a modification in their genome that results in a loss of expression of CD30, or expression of a variant form of CD30 that is not recognized by an immunotherapeutic agent targeting CD30, is created using a nuclease and/or a gRNA targeting human CD30 as described herein. It will be understood that such a cell can be created by contacting the cell with the nuclease and/or the gRNA, or the cell can be the daughter cell of a cell that was contacted with the nuclease and/or gRNA. In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells. In some preferred embodiments, a genetically engineered hematopoietic cell provided herein, or its progeny, can differentiate into all blood cell lineages, preferably without any differentiation bias as compared to a hematopoietic cell of the same cell type, but not comprising a genomic modification that results in a loss of expression of CD30, or expression of a variant form of CD30 that is not recognized by an immunotherapeutic agent targeting CD30.

It will be understood that, upon engrafting donor cells into a recipient host organism, the relative levels of the engrafted donor cells (and descendants thereof) and the host cells, e.g., in a given niche (e.g., bone marrow), are important for physiological and/or therapeutic outcomes for the host organism. The level of engrafted donor cells or descendants thereof relative to host cells in a given tissue or niche is referred to herein as chimerism. In some embodiments, a cell described herein (e.g., an HSC or HPC) is capable of engrafting in a human subject and does not exhibit any difference in chimerism as compared to a hematopoietic cell of the same cell type, but not comprising a genomic modification that results in a loss of expression of CD30, or expression of a variant form of CD30 that is not recognized by an immunotherapeutic agent targeting CD30. In some embodiments, a cell described herein (e.g., an HSC or HPC) is capable of engrafting in a human subject exhibits no more than a 1, 2, 5, 10, 15, 20, 25, 30, 35, 40, 45, or 50% difference in chimerism as compared to a hematopoietic cell of the same cell type, but not comprising a genomic modification that results in a loss of expression of CD30, or expression of a variant form of CD30 that is not recognized by an immunotherapeutic agent targeting CD30.

In some embodiments, a genetically engineered cell provided herein comprises only one genomic modification, e.g., a genomic modification that results in a loss of expression of CD30, or expression of a variant form of CD30 that is not recognized by an immunotherapeutic agent targeting CD30. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in a loss of expression of CD30, or expression of a variant form of CD30 that is not recognized by an immunotherapeutic agent targeting CD30.

In some embodiments, a genetically engineered cell provided herein comprises a genomic modification that results in a loss of expression of CD30, or expression of a variant form of CD30 that is not recognized by an immunotherapeutic agent targeting CD30, and further comprises an expression construct that encodes a chimeric antigen receptor, e.g., in the form of an expression construct encoding the CAR integrated in the genome of the cell. In some embodiments, the CAR comprises a binding domain, e.g., an antibody fragment, that binds CD30.

Some aspects of this disclosure provide genetically engineered immune effector cells comprising a modification in their genome that results in a loss of expression of CD30, or expression of a variant form of CD30 that is not recognized by an immunotherapeutic agent targeting CD30. In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell. In some embodiments, the immune effector cell does not express an endogenous transgene, e.g., a transgenic protein. In some embodiments, the immune effector cell expresses a chimeric antigen receptor (CAR). In some embodiments, the immune effector cell expresses a CAR targeting CD30. In some embodiments, the immune effector cell does not express a CAR targeting CD30.

In some embodiments, a genetically engineered cell provided herein comprises a genomic modification that results in a loss of expression of CD30, or expression of a variant form of CD30 that is not recognized by an immunotherapeutic agent targeting CD30, and does not comprise an expression construct that encodes an exogenous protein, e.g., does not comprise an expression construct encoding a CAR.

In some embodiments, a genetically engineered cell provided herein expresses substantially no CD30 protein, e.g., expresses no CD30 protein that can be measured by a suitable method, such as an immunostaining method. In some embodiments, a genetically engineered cell provided herein expresses substantially no wild-type CD30 protein, but expresses a mutant CD30 protein variant, e.g., a variant not recognized by an immunotherapeutic agent targeting CD30, e.g., a CAR-T cell therapeutic, or an anti-CD30 antibody, antibody fragment, or antibody-drug conjugate (ADC).

In some embodiments, the genetically engineered cells provided herein are hematopoietic cells, e.g., hematopoietic stem cells, hematopoietic progenitor cells (HPCs), hematopoietic stem or progenitor cells. Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of one or more cell surface markers, e.g., CD34 (e.g., CD34+), which can be used for the identification and/or isolation of HSCs, and absence of cell surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells.

In some embodiments, the genetically engineered HSCs are obtained from a subject, such as a human subject. Methods of obtaining HSCs are described, e.g., in PCT Application No. US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g. heterogeneous population of genetically engineered cells containing different CD30 mutations. In some embodiments, at least 40%, at least 50%, at least 60%, at least 70%, at least 75%, at least 80%, at least 85%, at least 90%, or at least 95% of copies of a gene encoding CD30 in the population of genetically engineered cells comprise a mutation effected by a genome editing approach described herein, e.g., by a CRISPR/Cas system using a gRNA provided herein. By way of example, a population of genetically engineered cells can comprise a plurality of different CD30 mutations and each mutation of the plurality may contribute to the percent of copies of CD30 in the population of cells that have a mutation.

In some embodiments, the expression of CD30 on the genetically engineered hematopoietic cell is compared to the expression of CD30 on a naturally occurring hematopoietic cell (e.g., a wild-type counterpart). In some embodiments, the genetic engineering results in a reduction in the expression level of CD30 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of CD30 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD30 as compared to a naturally occurring hematopoietic cell (e.g., a wild-type counterpart).

In some embodiments, the genetic engineering as described herein, e.g., using a gRNA targeting CD30 as described herein, results in a reduction in the expression level of wild-type CD30 by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the level of wild-type CD30 on a naturally occurring hematopoietic cell (e.g., a wild-type counterpart). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of CD30 as compared to a naturally occurring hematopoietic cell (e.g., a wild-type counterpart).

In some embodiments, the genetic engineering as described herein, e.g., using a gRNA targeting CD30 as described herein, results in a reduction in the expression level of wild-type lineage-specific cell surface antigen (e.g., CD30) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to a suitable control (e.g., a cell or plurality of cells). In some embodiments, the suitable control comprises the level of the wild-type lineage-specific cell surface antigen measured or expected in a plurality of non-engineered cells from the same subject. In some embodiments, the suitable control comprises the level of the wild-type lineage-specific cell surface antigen measured or expected in a plurality of cells from a healthy subject. In some embodiments, the suitable control comprises the level of the wild-type lineage-specific cell surface antigen measured or expected in a population of cells from a pool of healthy individuals (e.g., 10, 20, 50, or 100 individuals). In some embodiments, the suitable control comprises the level of the wild-type lineage-specific cell surface antigen measured or expected in a subject in need of a treatment described herein, e.g., an anti-CD30 therapy, e.g., wherein the subject has a cancer, wherein cells of the cancer express CD30.

In some embodiments, a method of genetically engineering cells described herein comprises a step of providing a wild-type cell, e.g., a wild-type hematopoietic stem or progenitor cell. In some embodiments, the wile-type cell is an un-edited cell comprising (e.g., expressing) two functional copies of a gene encoding CD30. In some embodiments, the cell comprises a CD30 gene sequence according to SEQ ID NO: 113. In some embodiments, the cell comprises a CD30 gene sequence encoding a CD30 protein that is encoded in SEQ ID NO: 113, e.g., the CD30 gene sequence may comprise one or more silent mutations relative to SEQ ID NO: 113. In some embodiments, the cell used in the method is a naturally occurring cell or a non-engineered cell. In some embodiments, the wild-type cell expresses CD30, or gives rise to a more differentiated cell that expresses CD30 at a level comparable to (or within 90%-110%, 80%-120%, 70%-130%, 60-140%, or 50%-150% of) a cell line expressing CD30, such as B cell-derived Hodgkin’s cell lines (e.g., L1236, L428, KM-H2, and L591), ALCL cell lines, among others.

In some embodiments, the wild-type cell binds an antibody that binds CD30 (e.g., an anti-CD30 antibody), or gives rise to a more differentiated cell that binds such an antibody at a level comparable to (or within 90%-110%, 80%-120%, 70%-130%, 60-140%, or 50%-150% of) binding of the antibody to a cell line expressing CD30, e.g., L1236, L428, KM-H2, and L591). Antibody binding may be measured, for example, by flow cytometry or immunohistochemistry.

### Dual gRNA Compositions and Uses Thereof

In some embodiments, a gRNA provided herein (e.g., a gRNA provided in any of Tables 1-4) can be used in combination with a second gRNA, e.g., for targeting a CRISPR/Cas nuclease to two sites in a genome. For instance, in some embodiments it may desired to produce a hematopoietic cell that is deficient for CD30 and a second lineage-specific cell surface antigen, e.g., CD33, CD123, CLL-1, CD19, CD30, CD5, CD6, CD7, CD38, or BCMA, so that the cell can be resistant to two agents: an anti-CD30 agent and an agent targeting the second lineage-specific cell surface antigen. In some embodiments, it is desirable to contact a cell with two different gRNAs that target different sites of CD30, e.g., in order to make two cuts and create a deletion or an insertion between the two cut sites. Accordingly, the disclosure provides various combinations of gRNAs and related CRISPR systems, as well as cells created by genome editing methods using such combinations of gRNAs and related CRISPR systems. In some embodiments, the CD30 gRNA binds a different nuclease than the second gRNA. For example, in some embodiments, the CD30 gRNA may bind Cas9 and the second gRNA may bind Cas12a, or vice versa.

In some embodiments, the first gRNA is a CD30 gRNA provided herein (e.g., a gRNA provided in any of Tables 1-4 or a variant thereof) and the second gRNA targets a lineage-specific cell-surface antigen chosen from: BCMA, CD19, CD20, CD30, ROR1, B7H6, B7H3, CD23, CD33, CD38, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD34, CD14, CD66b, CD41, CD61, CD62, CD235a, CD146, CD326, LMP2, CD22, CD52, CD10, CD3/TCR, CD79/BCR, and CD26.

In some embodiments, the first gRNA is a CD30 gRNA provided herein (e.g., a gRNA provided in any one of Tables 1-4 or a variant thereof) and the second gRNA targets a lineage-specific cell-surface antigen associated with a neoplastic or malignant disease or disorder, e.g., with a specific type of cancer, such as, without limitation, CD20, CD22 (Non-Hodgkin’s lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the first gRNA is a CD30 gRNA provided herein (e.g., a gRNA provided in any one of Tables 1-4 or a variant thereof) and the second gRNA targets a lineage-specific cell-surface antigen chosen from: CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD14, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD272, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, CD306, CD307a, CD307b, CD307c, CD307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 or CD363.

In some embodiments, the second gRNA is a gRNA disclosed in any of WO2017/066760, WO2019/046285, WO/2018/160768, or Borot et al. PNAS (2019) 116 (24):11978-11987, each of which is incorporated herein by reference in its entirety.

### Methods of Administration to Subjects in Need Thereof

Some aspects of this disclosure provide methods comprising administering an effective number of genetically engineered cells as described herein, comprising a modification in their genome that results in a loss of expression of CD30, or expression of a variant form of CD30 that is not recognized by an immunotherapeutic agent targeting CD30, to a subject in need thereof.

A subject in need thereof is, in some embodiments, a subject undergoing or about to undergo an immunotherapy targeting CD30. A subject in need thereof is, in some embodiments, a subject having or having been diagnosed with, a malignancy characterized by expression of CD30 on malignant cells. In some embodiments, a subject having such a malignancy may be a candidate for immunotherapy targeting CD30, but the risk of detrimental on-target, off-disease effects may outweigh the benefit, expected or observed, to the subject. In some such embodiments, administration of genetically engineered cells as described herein, results in an amelioration of the detrimental on-target, off-disease effects, as the genetically engineered cells provided herein are not targeted efficiently by an immunotherapeutic agent targeting CD30.

In some embodiments, the malignancy is a hematologic malignancy, or a cancer of the blood. In some embodiments, the malignancy is a lymphoid malignancy. In general, lymphoid malignancies are associated with the inappropriate production, development, and/or function of lymphoid cells, such as lymphocytes of the T lineage or the B lineage. In some embodiments, the malignancy is characterized or associated with cells that express CD30 on the cell surface.

As described herein, CD30 is a marker for a T cell lymphoma, B cell lymphoma, or an NK cell lymphoma. See, e.g., Sabattini et al. Haematologica (2013) 98(8): e81-e82; Grover et al. BMC Cancer (2019) 19: 203.

In some embodiments, the malignancy is associated with aberrant T lymphocytes, such as a T-lineage cancer, e.g., a T cell leukemia or a T-cell lymphoma.

Examples of T cell leukemias and T-cell lymphomas include, without limitation, T-lineage Acute Lymphoblastic Leukemia (T-ALL), Hodgkin’s lymphoma, or a non-Hodgkin’s lymphoma, acute lymphoblastic leukemia (ALL), chronic lymphocytic leukemia (CLL), large granular lymphocytic leukemia, adult T-cell leukemia/lymphoma (ATLL), T-cell prolymphocytic leukemia (T-PLL), T-cell chronic lymphocytic leukemia,, T-prolymphocytic leukemia, T-cell lymphocytic leukemia, peripheral T-cell lymphoma not otherwise specified (PTCL-NOS), enteropathy associated T-cell lymphoma, B-cell chronic lymphocytic leukemia, mantle cell lymphoma, peripheral T-cell lymphoma (PTCL), anaplastic large-cell lymphoma, cutaneous T-cell lymphoma, angioimmunoblastic lymphoma, anaplastic large cell lymphoma, enteropathy-type T-cell lymphoma, hematosplenic gamma-delta T-cell lymphoma, lymphoblastic lymphoma, or hairy cell leukemia.

In some embodiments, the malignancy is associated with aberrant B lymphocytes, such as a B-lineage cancer, e.g., a B-cell leukemia or a B-cell lymphoma. In some embodiments, the malignancy is B-lineage Acute Lymphoblastic Leukemia (B-ALL) or chronic lymphocytic leukemia (B-CLL), primary mediastinal B-cell lymphoma.

In some embodiments, the malignancy is a neoplasm, such as a cutaneous CD30-positive lymphoproliferative disorder (e.g., primary cutaneous ALCL, lymphomatoid papulosis), mycosis fungoides, nonepidermotropic cutaneous T cell lymphoma, or reactive cutaneous lymphoproliferations.

In some embodiments, the malignancy is graft-versus host disease.

Also within the scope of the present disclosure are malignancies that are considered to be relapsed and/or refractory, such as relapsed or refractory hematological malignancies. A subject in need thereof is, in some embodiments, a subject undergoing or that will undergo an immune effector cell therapy targeting CD30, e.g., CAR-T cell therapy, wherein the immune effector cells express a CAR targeting CD30, and wherein at least a subset of the immune effector cells also express CD30 on their cell surface. As used herein, the term “fratricide” refers to self-killing. For example, cells of a population of cells kill or induce killing of cells of the same population. In some embodiments, cells of the immune effector cell therapy kill or induce killing of other cells of the immune effector cell therapy. In such embodiments, fratricide ablates a portion of or the entire population of immune effector cells before a desired clinical outcome, e.g., ablation of malignant cells expressing CD30 within the subject, can be achieved. In some such embodiments, using genetically engineered immune effector cells, as provided herein, e.g., immune effector cells that do not express CD30 or do not express a CD30 variant recognized by the CAR, as the immune effector cells forming the basis of the immune effector cell therapy, will avoid such fratricide and the associated negative impact on therapy outcome. In such embodiments, genetically engineered immune effector cells, as provided herein, e.g., immune effector cells that do not express CD30 or do not express a CD30 variant recognized by the CAR, may be further modified to also express the CD30-targeting CAR. In some embodiments, the immune effector cells may be lymphocytes, e.g., T-lymphocytes, such as, for example alpha/beta T-lymphocytes, gamma/delta T-lymphocytes, or natural killer T cells. In some embodiments, the immune effector cells may be natural killer (NK) cells.

In some embodiments, an effective number of genetically engineered cells as described herein, comprising a modification in their genome that results in a loss of expression of CD30, or expression of a variant form of CD30 that is not recognized by an immunotherapeutic agent targeting CD30, is administered to a subject in need thereof, e.g., to a subject undergoing or that will undergo an immunotherapy targeting CD30, wherein the immunotherapy is associated or is at risk of being associated with a detrimental on-target, off-disease effect, e.g., in the form of cytotoxicity towards healthy cells in the subject that express CD30. In some embodiments, an effective number of such genetically engineered cells may be administered to the subject in combination with the anti-CD30 immunotherapeutic agent.

It is understood that when agents (e.g., CD30-modified cells and an anti-CD30 immunotherapeutic agent) are administered in combination, the cells and the agent may be administered at the same time or at different times, e.g., in temporal proximity. Furthermore, the cells and the agent may be admixed or in separate volumes or dosage forms. For example, in some embodiments, administration in combination includes administration in the same course of treatment, e.g., in the course of treating a subject with an anti-CD30 immunotherapy, the subject may be administered an effective number of genetically engineered, CD30-modified cells concurrently or sequentially, e.g., before, during, or after the treatment, with the anti-CD30 immunotherapy.

In some embodiments, the immunotherapeutic agent that targets CD30 as described herein is an immune cell that expresses a chimeric antigen receptor, which comprises an antigen-binding fragment (e.g., a single-chain antibody) capable of binding to CD30. The immune cell may be, e.g., a T cell (e.g., a CD4+ or CD8+ T cell) or an NK cell.

A Chimeric Antigen Receptor (CAR) can comprise a recombinant polypeptide comprising at least an extracellular antigen binding domain, a transmembrane domain, and a cytoplasmic signaling domain comprising a functional signaling domain, e.g., one derived from a stimulatory molecule. In one some embodiments, the cytoplasmic signaling domain further comprises one or more functional signaling domains derived from at least one costimulatory molecule, such as 4-1BB (i.e., CD137), CD27, and/or CD28, or fragments of those molecules. The extracellular antigen binding domain of the CAR may comprise a CD30-binding antibody fragment. The antibody fragment can comprise one or more CDRs, the variable regions (or portions thereof), the constant regions (or portions thereof), or combinations of any of the foregoing.

Amino acid and nucleic acid sequences of an exemplary heavy chain variable region and light chain variable region of an anti-human CD30 antibody are provided, for example in PCT Publication Nos. WO 2003/059282, WO 2005/001038, U.S. Publication No. 2007/0166309.

A chimeric antigen receptor (CAR) typically comprises an antigen-binding domain, e.g., comprising an antibody fragment, fused to a CAR framework, which may comprise a hinge region (e.g., from CD8 or CD28), a transmembrane domain (e.g., from CD8 or CD28), one or more costimulatory domains (e.g., CD28 or 4-1BB), and a signaling domain (e.g., CD3zeta). Exemplary sequences of CAR domains and components are provided, for example in PCT Publication No. WO 2019/178382, and in Table 5 below.

TABLE 5

Exemplary components of a chimeric receptor

| | |
|---|---|
| Chimeric receptor component | Amino acid sequence |
| Antigen-binding fragment | Light chain- Linker-Heavy chain |
| CD28 costimulatory domain | IEVMYPPPYLDNEKSNGTIIHVKGKHLCP SPLFPGPSKPFWVLVVVGGVLACYSLLVTV AFIIFWVRSKRSRLLHSDYMNMTPRRPGPT RKHYQPYAPPRDFAAYRS (SEQ ID NO: 114) |
| CD8alpha transmembrane domain | IYIWAPLAGTCGVLLLSLVITLYC (SEQ ID NO: 115) |
| CD28 transmembrane domain | FWVLVVVGGVLACYSLLVTVAFII FWVRSKRSRLLHSDYMNMTPRR PGPTRKHYQPYAPPRDFAAYRS (SEQ ID NO: 116) |
| 4-1BB intracellular domain | KRGRKKLLYIFKQPFMRVQTTQEEDGCS CRFPEEEEGGCEL (SEQ ID NO: 117) |
| CD3ζ cytoplasmic signaling domain | RVKFSRSADAPAYQQGQNQLYNELNLG RREEYDVLDKRRGRDPEMGGKPQRRKNP QEGLYNELQKDKMAEAYSEIGMKGERRR GKGHDGLYQGLSTATKDTYDALHMQALPPR (SEQ ID NO: 118) |

In some embodiments, the number of genetically engineered cells provided herein, e.g., HSCs, HPCs, or immune effector cells that are administered to a subject in need thereof, is within the range of 10⁶-10¹¹. However, amounts below or above this exemplary range are also within the scope of the present disclosure. For example, in some embodiments, the number of genetically engineered cells provided herein, e.g., HSCs, HPCs, or immune effector cells that are administered to a subject in need thereof is about 10⁶, about 10⁷, about 10⁸, about 10⁹, about 10¹⁰, or about 10¹¹. In some embodiments, the number of genetically engineered cells provided herein, e.g., HSCs, HPCs, or immune effector cells that are administered to a subject in need thereof, is within the range of 10⁶-10⁹, within the range of 10⁶-10⁸, within the range of 10⁷-10⁹, within the range of about 10⁷-10¹⁰, within the range of 10⁸-10¹⁰, or within the range of 10⁹-10¹¹.

In some embodiments, the immunotherapeutic agent that targets CD30 is an antibody-drug conjugate (ADC). The ADC may be a molecule comprising an antibody or antigen-binding fragment thereof conjugated to a toxin or drug molecule. Binding of the antibody or fragment thereof to the corresponding antigen allows for delivery of the toxin or drug molecule to a cell that presents the antigen on the its cell surface (e.g., target cell), thereby resulting in death of the target cell.

Suitable antibodies and antibody fragments binding CD30 will be apparent to those of ordinary skill in the art, and include, for example, SGN-30, a chimeric mouse-human antibody, the fully humanized mAb 5F11 (MDX-060, or iratumumab), the antibody-drug conjugate brentuximab vedotin (also referred to as SGN-35 or cAC10-vcMMAE), or those described in PCT Publication No. WO 2003/059282, WO 2005/001038, U.S. Publication No. 2007/0166309.

Toxins or drugs compatible for use in antibody-drug conjugates are known in the art and will be evident to one of ordinary skill in the art. See, e.g., Peters et al. Biosci. Rep.(2015) 35(4): e00225; Beck et al. Nature Reviews Drug Discovery (2017) 16:315-337; Marin-Acevedo et al. J. Hematol. Oncol. (2018)11: 8; Elgundi et al. Advanced Drug Delivery Reviews (2017) 122: 2-19.

In some embodiments, the antibody-drug conjugate may further comprise a linker (e.g., a peptide linker, such as a cleavable linker) attaching the antibody and drug molecule.

Examples of suitable toxins or drugs for antibody-drug conjugates include, without limitation, the toxins and drugs comprised in brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab vedotin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/ IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/ CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/ BAY1129980, aprutumab ixadotin/BAY1187982, ARX788, AGS62P1, XMT-1522, AbGn-107, MEDI4276, and DSTA4637S/RG7861. Anti-CD30 antibody drug conjugates are known in the art, for example, Bradley et al. Am. J. Health Syst. Pharm. (2013) 70(7): 589-97; Shen et al. mAbs (2019) 11(6): 1149-1161.

In some embodiments, binding of the antibody-drug conjugate to the epitope of the cell-surface lineage-specific protein induces internalization of the antibody-drug conjugate, and the drug (or toxin) may be released intracellularly. In some embodiments, binding of the antibody-drug conjugate to the epitope of a cell-surface lineage-specific protein induces internalization of the toxin or drug, which allows the toxin or drug to kill the cells expressing the lineage-specific protein (target cells). In some embodiments, binding of the antibody-drug conjugate to the epitope of a cell-surface lineage-specific protein induces internalization of the toxin or drug, which may regulate the activity of the cell expressing the lineage-specific protein (target cells). The type of toxin or drug used in the antibody-drug conjugates described herein is not limited to any specific type.

Some of the embodiments, advantages, features, and uses of the technology disclosed herein will be more fully understood from the Examples below. The Examples are intended to illustrate some of the benefits of the present disclosure and to describe particular embodiments but are not intended to exemplify the full scope of the disclosure and, accordingly, do not limit the scope of the disclosure.

### EXAMPLES

### Example 1: Genetic Editing of CD30 in Human Cells

### Design of RNA Constructs

The target domains and gRNAs indicated in Tables 1-4 were designed by manual inspection for a PAM sequence for an applicable nuclease, e.g., Cas9, Cpf1, with close proximity to the target region and prioritized according to predicted specificity by minimizing potential off-target sites in the human genome with an online search algorithm (e.g., the Benchling algorithm, Doench et al 2016, Hsu et al 2013). All designed synthetic sgRNAs were produced with chemically modified nucleotides at the three terminal positions at both the 5′ and 3′ ends. Modified nucleotides contained 2′-O-methyl-3′-phosphorothioate (abbreviated as “ms”) and the ms-sgRNAs were HPLC-purified

### Editing in Primary Human CD34+ HSCs

Frozen CD34+ HSCs derived from mobilized peripheral blood (mPB) are purchased, for example, from Hemacare or Fred Hutchinson Cancer Center and thawed according to manufacturer’s instructions. To edit HSCs, ~1×10⁶ HSCs are thawed and cultured in StemSpan SFEM medium supplemented with StemSpan CC110 cocktail (StemCell Technologies) for 24-48 h before electroporation with RNP. To electroporate HSCs, 1.5 ×10⁵ cells are pelleted and resuspended in 20 µL Lonza P3 solution and mixed with 10 µL Cas9 RNP. CD34+ HSCs are electroporated using the Lonza Nucleofector 2 and the Human P3 Cell Nucleofection Kit (VPA-1002, Lonza). Cas9 protein is purchased from Synthego.

### Genomic DNA Analysis

For all genomic analysis, DNA is harvested from cells, amplified with primers flanking the target region, purified and the allele modification frequencies are analyzed using TIDE (Tracking of Indels by Decomposition). Analyses are performed using a reference sequence from a mock-transfected sample. Parameters are set to the default maximum indel size of 10 nucleotides and the decomposition window to cover the largest possible window with high quality traces. All TIDE analyses below the detection sensitivity of 3.5% are set to 0%.

Human CD34+ cells are electroporated with Cas9 protein and indicated CD30-targeting gRNAs, as described above. The percentage editing is determined by % INDEL as assessed by TIDE analysis. Editing efficiency is determined by flow cytometric analysis.

### Flow Cytometry Analysis

The CD30 gRNA-edited cells may also be evaluated for surface expression of CD30 protein, for example by flow cytometry analysis (FACS). Live CD34+ HSCs are stained for CD30 using an anti-CD30 antibody and analyzed by flow cytometry on the Attune NxT flow cytometer (Life Technologies). Cells in which the CD30 gene have been genetically modified show a reduction in CD30 expression as detected by FACS.

### Viability of Edited Cells

At 4, 24, and 48 hours post-ex vivo editing, the percentages of viable, edited CD30KO cells and control cells are quantified using flow cytometry and the 7AAD viability dye. CD30KO cells edited using the CD30 gRNAs described herein may be viable and remain viable over time following electroporation and gene editing. This is similar to what is observed in the control mock edited cells.

### Example 2: Genetic Editing of T-Lymphocytes

CD30 gRNAs were designed as described in Example 1 and shown in Tables 1-4. To assess editing efficiency in B cell-derived Hodgkin’s cell lines (e.g., L1236, L428, KM-H2, and L591), the cells are electroporated with pre-formed gRNA-nuclease (e.g., Cas9, Cpfl) RNP complex. Briefly, approximated 2e5 Molt-4 cells are electroporated with 3 ug Cas9:3 ug gRNA preformed RNP complex using a Lonza 4D-Nucleofector and P3 Primary Cell Kit.

At 48 hours post electroporation, the editing frequency is determined based on the percentage of alleles with indels compared to the wild-type sequence as assessed by Sanger sequence, followed by Tracking of Indels by Decomposition (TIDE) analysis (see, Brinkman et al. 2014; Hsiau et al. 2018).

The percentage editing is determined by % INDEL as assessed by TIDE. Editing efficiency is also evaluated based on expression of CD30 by flow cytometric analysis. This process demonstrates a reduction in CD30 protein detected in cells edited using the CD30 gRNAs.

### Example 3: CAR-T Cytotoxicity Assay

Genetically modified cells produced using the gRNAs shown in Tables 1-4 may be evaluated for killing by CD30-CAR T cells.

### CAR Constructs and Lentiviral Production

Second-generation CARs are constructed to target CD30. An exemplary CAR construct consists of an extracellular scFv antigen-binding domain, using CD8α signal peptide, CD8α hinge and transmembrane regions, the 4-1BB costimulatory domain, and the CD3ξ signaling domain. The anti-CD30 scFv sequence may be obtained from any anti-CD30 antibody known in the art, such as SGN-30, mAb 5F11 (MDX-060 or iratumumab), brentuximab vedotin or any other anti-CD30 antibodies referenced herein. CAR cDNA sequences for the target are sub-cloned into the multiple cloning site of the pCDH-EF1α-MCS-T2A-GFP expression vector, and lentivirus is generated following the manufacturer’s protocol (System Biosciences). Lentivirus can be generated by transient transfection of 293TN cells (System Biosciences) using Lipofectamine 3000 (ThermoFisher). The exemplary CAR construct is generated by cloning the light and heavy chain of an anti-CD30 antibody, to the CD8α hinge domain, the ICOS transmembrane domain, the ICOS signaling domain, the 4-1BB signaling domain and the CD3ξ signaling domain into the lentiviral plasmid pHIV-Zsgreen.

### CAR Transduction and Expansion

Human primary T cells are isolated from Leuko Pak (Stem Cell Technologies) by magnetic bead separation using anti-CD4 and anti-CD8 microbeads according to the manufacturer’s protocol (Stem Cell Technologies). Purified CD4+ and CD8+ T cells are mixed 1:1 and activated using anti-CD3/CD28 coupled Dynabeads (Thermo Fisher) at a 1:1 bead to cell ratio. T cell culture media used is CTS Optimizer T cell expansion media supplemented with immune cell serum replacement, L-Glutamine and GlutaMAX (all purchased from Thermo Fisher) and 100 IU/mL of IL-2 (Peprotech). T cell transduction is performed 24 hours post activation by spinoculation in the presence of polybrene (Sigma). CAR-T cells are cultured for 9 days prior to cryopreservation. Prior to all experiments, T cells are thawed and rested at 37° C. for 4-6 hours.

### Flow Cytometry Based CAR-T Cytotoxicity Assay

The cytotoxicity of target cells is measured by comparing survival of target cells relative to the survival of negative control cells. For CD30 cytotoxicity assays, wildtype and CRISPR/Cas9 edited cells of a CD30-expressing B cell-derived Hodgkin’s cell line, such as L1236, L428 cells, are used as target cells. Wildtype Raji cell lines (ATCC) are used as negative control for both experiments. Alternatively, CD34+ cells may be used as target cells and CD34+ cells deficient in CD30 or having reduced expression of CD30 may be generated as described in Example 1.

Target cells and negative control cells are stained with CellTrace Violet (CTV) and CFSE (Thermo Fisher), respectively, according to the manufacturer’s instructions. After staining, target cells and negative control cells are mixed at 1:1.

Anti-CD30 CAR-T cells are used as effector T cells. Non-transduced T cells (mock CAR-T) are used as control. The effector T cells are co-cultured with the target cell/negative control cell mixture at a 1:1 effector to target ratio in duplicate. A group of target cell/negative control cell mixture alone without effector T cells is included as control. Cells are incubated at 37° C. for 24 hours before flow cytometric analysis. Propidium iodide (ThermoFisher) is used as a viability dye. For the calculation of specific cell lysis, the fraction of live target cell to live negative control cell (termed target fraction) is used. Specific cell lysis is calculated as ((target fraction without effector cells - target fraction with effector cells)/(target fraction without effectors)) × 100%.

### Example 4: Effect of Anti-CD30 Antibody Drug Conjugates on Engineered HSCs

Genetically modified cells produced using the gRNAs shown in Tables 1-4 may be evaluated for killing by antibody-drug conjugates, such as an anti-CD30 antibody (e.g., brentuximab vedotin) conjugated to an immunotoxin.

Frozen CD34+ HSPCs derived from mobilized peripheral blood are thawed and cultured for 72 h before electroporation with ribonucleoprotein comprising Cas9 and an sgRNA. Samples are electroporated with the following conditions:
i. Mock (Cas9 only), and
ii. KO sgRNA (such as any one of the CD30 gRNAs shown in Tables 1-4). Cells are allowed to recover for 72 hours and genomic DNA is collected and analyzed. The percentage of CD30-positive cells is assessed by flow cytometry to confirm that editing with the CD30 gRNAs is effective in knocking out or reducing CD30. The editing events in the HSCs result in a variety of indel sequences.

### Sensitivity of Cells Having CD30 Deletion to Antibody-Drug Conjugates

To determine in vitro toxicity, cells are incubated with the antibody-drug conjugate in the culture media and the number of viable cells is quantified over time. Engineered cells that are deficient in CD30 or have reduced CD30 expression generated with the CD30 gRNAs described herein are more resistant to antibody-drug conjugate treatment than cells expressing full length CD30 (mock).

### (ii) Enrichment of CD30-Modified Cells

To assay if CD30-modified cells are enriched following treatment with the antibody-drug conjugate, CD34+ HSPCs are edited with 50% of standard nuclease (e.g., Cas9, Cpf1) to gRNA ratios. The bulk population of cells are analyzed prior to and after treatment with the antibody-drug conjugate. Following treatment with the antibody-drug conjugate, CD30-modified cells are enriched so that the percentage of CD30-deficient cells increased.

### (iii) In Vitro Differentiation of CD34+ HSPCs

Cell populations are assessed for lymphoid differentiation prior to and after treatment with the antibody-drug conjugate at various days post differentiation. Engineered CD30 knockout cells generated with the CD30 gRNAs described herein may show increased expression of lymphoid differentiation markers, whereas cells expressing full length CD30 (mock) may not differentiate.

### Example 5: Evaluation of the Persistence of CD30KO CD34+ Cells In Vivo

### Editing in Mobilized Peripheral Blood CD34+ HSCs (mPB CD34+HSPCs)

gRNAs (Synthego) were designed as described in Example 1. mPB CD34+ HSPCs are purchased from Fred Hutchinson Cancer Center and thawed according to manufacturer’s instructions. These cells are then edited via CRISPR/Cas9 as described in Example 1 using the CD30-targeting gRNAs described herein, as well as a non-CD30 targeting control gRNA (gCtrl) that is designed not to target any region in the human or mouse genomes.

At 4, 24, and 48 hours post-ex vivo editing, the percentages of viable, edited CD30KO cells and control cells are quantified using flow cytometry and the 7AAD viability dye. High levels of CD30KO cells edited using the CD30 gRNAs described herein may be viable and remain viable over time following electroporation and gene editing, comparable to what is observed in the control cells edited with the non-CD30 targeting control gRNA, gCtrl.

Additionally, at 48 hours post-ex vivo editing, the genomic DNA is harvested from cells, PCR amplified with primers flanking the target region, purified, and analyzed by TIDE, in order to determine the percentage editing as assessed by INDEL (insertion/deletion), as described in Example 1.

Following TIDE analysis, the percentage of long term-HSCs (LT-HSCs) following editing with the CD30 gRNAs described herein are quantified by flow cytometry. The percentages of LT-HSCs following editing with the specified CD30 gRNAs is assessed. This assay may be performed, for example, at the time of cryopreservation of the edited cells, prior to injection into mice for investigation of persistence of CD30KO cells in vivo. The edited cells are cryopreserved in CryoStor® CS10 media (Stem Cell Technology) at 5×10⁶ cells/mL, in a 1 mL volume of media per vial.

### Investigating Engraftment Efficiency and Persistence of CD30KO mPB CD34+ HSPCs In Vivo

Female NSG mice (JAX) that are 6 to 8 weeks of age, are allowed to acclimate for 2-7 days. Following acclimation, mice are irradiated using 175 cGy whole body irradiation by X-ray irradiator. This is regarded as day 0 of the investigation. At 4-10 hours, following irradiation, the mice are engrafted with the CD30KO cells generated during any of the CD30 gRNAs described herein or control cells edited with gCtrl. The cryopreserved cells are thawed and counted using a BioRad TC-20 automated cell counter. The number of viable cells is quantified in the thawed vials, which is used to prepare the total number of cells for engraftment in the mice. Mice are given a single intravenous injection of 1×10⁶ edited cells in a 100 µL volume. Body weight and clinical observations are recorded once weekly for each mouse in the four groups.

At weeks 8 and 12 following engraftment, 50 µL of blood is collected from each mouse by retroorbital bleed for analysis by flow cytometry. At week 16, following engraftment, mice are sacrificed, and blood, spleens, and bone marrow are collected for analysis by flow cytometry. Bone marrow is isolated from the femur and the tibia. Bone marrow from the femur is also used for on-target editing analysis. Flow cytometry is performed using the FACSCanto™ 10 color and BDFACSDiva™ software. Cells are generally first sorted by viability using the 7AAD viability dye (live/dead analysis), then Live cells are gated by expression of human CD45 (hCD45) but not mouse CD45 (mCD45). The cells that are hCD45+ are then further gated for the expression of human CD19 (hCD19) (lymphoid cells, specifically B cells). Cells expressing human CD45 (hCD45) are also gated and analyzed for the presence of for various cellular markers of the myeloid lineage.

Numbers of cells expressing each of the analyzed markers that are comparable across all mice regardless of which edited cells they are engrafted with indicates successful engraftment of CD30KO cells edited with the CD30 gRNAs in the blood of mice.

At weeks 8, 12, and 16 following engraftment, the percentage of nucleated blood cells that are hCD45+ is quantified in the groups of mice (n=15 mice/group) that received control cells edited with the control gRNA (gCtrl), or the CD30KO cells. This is quantified by dividing the hCD45+ absolute cell count by the mouse CD45+ (mCD45) absolute cell count.

The percentage of hCD30+ cells in the blood is also quantified at week 8 following engraftment in the control and CD30KO mouse groups. Mice engrafted with the CD30KO cells (edited with any of the CD30 gRNAs described herein) are expected to have significantly lower levels of hCD30+ cells compared to the mice engrafted with control cells at weeks 8, 12, and 16.

Next, the percentages of particular populations of differentiated cells, such as CD19+ lymphoid cells, hCD14+ monocytes, and hCD11b+ granulocytes/neutrophils in the blood are quantified at weeks 8, 12, and 16 following engraftment in the mice engrafted with CD30KO cells or control cells. The levels of hCD19+ cells, hCD14+ cells, and hCD11b+ cells in the blood are equivalent between the control and CD30KO groups, and the levels of these cells remained equivalent from weeks 8 to 16 post-engraftment. Comparable levels of hCD19+, hCD14+, and hCD11b+ cells in the blood indicate that similar levels of human myeloid and lymphoid cell populations are present in mice that received the CD30KO cells and mice that received the control cells.

Finally, amplicon-seq may be performed on bone marrow samples isolated at week 16 post-engraftment to analyze the on-target CD30 editing in mice that are engrafted with the edited CD30KO cells.

### Evaluating Cell Samples Obtained From the Spleen of Engrafted Animals

At week 16 post-engraftment, the percentages of hCD45+ cells and the percentage of hCD30+ cells are also quantified in the spleen of mice that are engrafted with control cells or CD30KO cells. Comparable levels of hCD45+ cells and reduced levels of hCD30+ cells between the groups of mice (engrafted with control cells or CD30KO cells) may indicate the long-term persistence of CD30KO HSCs in the spleens of NSG mice.

Additionally, at week 16 post engraftment, the percentages of hCD14+ monocytes, hCD11b+ granulocytes/neutrophils, CD19+ lymphoid cells, and hCD3+ T cells in the spleen are quantified. Comparable levels of hCD14+ cells, hCD11b+ cells, hCD19+ cells, and hCD3+ in the spleen between the control and CD30KO groups may indicate that the edited CD30KO cells are capable of multilineage human hematopoietic cell reconstitution in the spleen of the NSG mice.

### Evaluating Blood and Bone Marrow Neutrophils

At week 16 post engraftment, the percentage of hCD11b+ cells are quantified in the blood and the bone marrow of mice engrafted with control cells or CD30KO cells. Comparable levels of CD11b+ neutrophil populations observed in the mice engrafted with control cells and the CD30KO cells in both the blood and the bone marrow of the NSG mice indicates successful engraftment and differentiation.

### Evaluating Blood and Bone Marrow Myeloid and Lymphoid Progenitor Cells

Also, at week 16, the percentage of hCD123+ cells in the blood and the percentage of hCD123+ cells in the bone marrow, and the percentage of hCD10+ cells in the bone marrow are quantified in mice engrafted with control cells or CD30KO cells. Comparable levels of myeloid and lymphoid progenitor cells between the control and CD30KO groups may indicate successful engraftment and development.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth explicitly herein.

What is claimed is:

1. A gRNA comprising a targeting domain, wherein the targeting domain comprises a sequence described in Tables 1-4.

2. A gRNA comprising a targeting domain, wherein the targeting domain comprises a sequence of any one of SEQ ID NOs: 55-81 and 102-111.

3. The gRNA of any of claims 1 and 2, wherein the gRNA comprises a first complementarity domain, a linking domain, a second complementarity domain which is complementary to the first complementarity domain, and a proximal domain.

4. The gRNA of any of claims 1-3, wherein the gRNA is a single guide RNA (sgRNA).

5. The gRNA of any of claims 1-4, wherein the gRNA comprises one or more nucleotide residues that are chemically modified.

6. The gRNA of any of claims 1-5, wherein the gRNA comprises one or more nucleotide residues that comprise a 2′O-methyl moiety.

7. The gRNA of any of claims 1-6, wherein the gRNA comprises one or more nucleotide residues that comprise a phosphorothioate.

8. The gRNA of any of claims 1-7, wherein the gRNA comprises one or more nucleotide residues that comprise a thioPACE moiety.

9. A method of producing a genetically engineered cell, comprising:
a. providing a cell, and
b. contacting the cell with (i) a gRNA of any of claims 1-8 or a gRNA targeting a targeting domain targeted by a gRNA of any one of claims 1-8; and (ii) an RNA-guided nuclease that binds the gRNA, thus forming a ribonucleoprotein (RNP) complex under conditions suitable for the gRNA of (i) to form and/or maintain an RNP complex with the RNA-guided nuclease of (ii) and for the RNP complex to bind a target domain in the genome of the cell.

10. The method of claim 9, wherein the RNA-guided nuclease is a CRISPR/Cas nuclease.

11. The method of claim 10, wherein the CRISPR/Cas nuclease is a Cas9 nuclease.

12. The method of claim 10, wherein the CRISPR/Cas nuclease is an spCas nuclease.

13. The method of claim 10, wherein the Cas nuclease in an saCas nuclease.

14. The method of claim 10, wherein the CRISPR/Cas nuclease is a Cpf1 nuclease.

15. The method of any one of claims 9-14, wherein the contacting comprises introducing (i) and (ii) into the cell in the form of a pre-formed ribonucleoprotein (RNP) complex.

16. The method of any one of claims 9-14, wherein the contacting comprises introducing (i) and/or (ii) into the cell in the form of a nucleic acid encoding the gRNA of (i) and/or the RNA-guided nuclease of (ii).

17. The method of any one of claims 9-14, wherein the nucleic acid encoding the gRNA of (i) and/or the RNA-guided nuclease of (ii) is an RNA, preferably an mRNA or an mRNA analog.

18. The method of any one of claims 9-15, wherein the ribonucleoprotein complex is introduced into the cell via electroporation.

19. The method of any one of claims 9-18, wherein the cell is a hematopoietic cell.

20. The method of any one of claims 9-19, wherein the cell is a hematopoietic stem cell.

21. The method of any one of claims 9-20, wherein the cell is a hematopoietic progenitor cell.

22. The method of any one of claims 9-18, wherein the cell is an immune effector cell.

23. The method of any one of claims 9-18 or 22, wherein the cell is a lymphocyte.

24. The method of any one of claims 9-18, 22, or 23, wherein the cell is a T-lymphocyte.

25. A genetically engineered cell, wherein the cell is obtained by the method of any of claims 9-24.

26. A cell population, comprising the genetically engineered cell of claim 25.

27. A cell population, comprising a genetically engineered cell, wherein the genetically engineered cell comprises a genomic modification that consists of an insertion or deletion immediately proximal to a site cut by an RNA-guided nuclease when bound to a gRNA comprising a targeting domain as described in any of Tables 1-4.

28. The cell population of claim 27, wherein the genomic modification is an insertion or deletion generated by a non-homologous end joining (NHEJ) event.

29. The cell population of claim 27, wherein the genomic modification is an insertion or deletion generated by a homology-directed repair (HDR) event.

30. The cell population of any one of claims 27-29, wherein the genomic modification results in a loss-of function of CD30 in a genetically engineered cell harboring such a genomic modification.

31. The cell population of any one of claims 27-30, wherein the genomic modification results in a reduction of expression of CD30 to less than 25%, less than 20% less than 10% less than 5% less than 2% less than 1%, less than 0.1%, less than 0.01%, or less than 0.001% as compared to the expression level of CD30 in wild-type cells of the same cell type that do not harbor a genomic modification of CD30.

32. The cell population of any one of claims 27-31, wherein the genetically engineered cell is a hematopoietic stem or progenitor cell.

33. The cell population of any one of claims 27-31, wherein the genetically engineered cell is an immune effector cell.

34. The cell population of any one of claims 27-31 or 33, wherein the genetically engineered cell is a T-lymphocyte.

35. The cell population of any one of claims 33 and 34, wherein the immune effector cell expresses a chimeric antigen receptor (CAR).

36. The cell population of claim 35, wherein the CAR targets CD30.

37. The cell population of any one of claims 26-32, which is characterized by the ability to engraft CD30-edited hematopoietic stem cells in the bone marrow of a recipient and to generate differentiated progeny of all blood lineage cell types in the recipient.

38. The cell population of any one of claims 26-32 or 37, which is characterized by the ability to engraft CD30-edited hematopoietic stem cells in the bone marrow of a recipient at an efficiency of at least 50%.

39. The cell population of any one of claims 26-32, 37, or 38, which is characterized by the ability to engraft CD30-edited hematopoietic stem cells in the bone marrow of a recipient at an efficiency of at least 60%.

40. The cell population of any one of claims 26-32 or 37-39, which is characterized by the ability to engraft CD30-edited hematopoietic stem cells in the bone marrow of a recipient at an efficiency of at least 70%.

41. The cell population of any one of claims 26-32 or 37-40, which is characterized by the ability to engraft CD30-edited hematopoietic stem cells in the bone marrow of a recipient at an efficiency of at least 80%.

42. The cell population of any one of claims 26-32 or 37-41, which is characterized by the ability to engraft CD30-edited hematopoietic stem cells in the bone marrow of a recipient at an efficiency of at least 90%.

43. The cell population of any of claims 26-32 or 37-42, wherein the cell population comprises CD30-edited hematopoietic stem cells that are characterized by a differentiation potential that is equivalent to the differentiation potential of non-edited hematopoietic stem cells.

44. A method, comprising administering to a subject in need thereof the genetically engineered cell of claim 25 or the cell population of any one of claims 26-43.

45. The method of claim 44, wherein the subject has or has been diagnosed with a hematopoietic malignancy.

46. The method of claim 44 or 45, further comprising administering to the subject an effective amount of an agent that targets CD30, wherein the agent comprises an antigen-binding fragment that binds CD30.


SEQUENCE LISTING (118 sequences)

SEQ ID NO: 1 - DNA, 20 bp, Artificial Sequence
  Synthetic
acatgccgggtcgacattcg

SEQ ID NO: 2 - DNA, 20 bp, Artificial Sequence
  Synthetic
cgtgtctgcgaatgtcgacc

SEQ ID NO: 3 - DNA, 20 bp, Artificial Sequence
  Synthetic
tcgacattcgcagacacggg

SEQ ID NO: 4 - DNA, 20 bp, Artificial Sequence
  Synthetic
gtgtcccttagacgacctcg

SEQ ID NO: 5 - DNA, 20 bp, Artificial Sequence
  Synthetic
gtcccttagacgacctcgtg

SEQ ID NO: 6 - DNA, 20 bp, Artificial Sequence
  Synthetic
gtcgacattcgcaggtgcgg

SEQ ID NO: 7 - DNA, 20 bp, Artificial Sequence
  Synthetic
cgaggcccttacctcgagaa

SEQ ID NO: 8 - DNA, 20 bp, Artificial Sequence
  Synthetic
ctgccaggatcgacccttcg

SEQ ID NO: 9 - DNA, 20 bp, Artificial Sequence
  Synthetic
acctcgagaacaagtcacgc

SEQ ID NO: 10 - DNA, 20 bp, Artificial Sequence
  Synthetic
cgtgacttgttctcgaggta

SEQ ID NO: 11 - DNA, 20 bp, Artificial Sequence
  Synthetic
catgccaggtcgacattcgc

SEQ ID NO: 12 - DNA, 20 bp, Artificial Sequence
  Synthetic
gggagccctcgtcagtttag

SEQ ID NO: 13 - DNA, 20 bp, Artificial Sequence
  Synthetic
cgtgagctgttctcgaggta

SEQ ID NO: 14 - DNA, 20 bp, Artificial Sequence
  Synthetic
acctcgagaacagctcacgc

SEQ ID NO: 15 - DNA, 20 bp, Artificial Sequence
  Synthetic
gcgtgacttgttctcgaggt

SEQ ID NO: 16 - DNA, 20 bp, Artificial Sequence
  Synthetic
agcccgactactacctggac

SEQ ID NO: 17 - DNA, 20 bp, Artificial Sequence
  Synthetic
gcccgtcacccgcttacctg

SEQ ID NO: 18 - DNA, 20 bp, Artificial Sequence
  Synthetic
ttctccacgaggtcgtctaa

SEQ ID NO: 19 - DNA, 20 bp, Artificial Sequence
  Synthetic
gaggaggacgcgcatccccg

SEQ ID NO: 20 - DNA, 20 bp, Artificial Sequence
  Synthetic
gcctgcgtgacttgttctcg

SEQ ID NO: 21 - DNA, 20 bp, Artificial Sequence
  Synthetic
cacacggcgtcttctccacg

SEQ ID NO: 22 - DNA, 20 bp, Artificial Sequence
  Synthetic
aaggcccttacctcgagaac

SEQ ID NO: 23 - DNA, 20 bp, Artificial Sequence
  Synthetic
aagggtcgatcctggcaggt

SEQ ID NO: 24 - DNA, 20 bp, Artificial Sequence
  Synthetic
caggtcgacattcgcaggtg

SEQ ID NO: 25 - DNA, 20 bp, Artificial Sequence
  Synthetic
aatcatccctgccggacaga

SEQ ID NO: 26 - DNA, 20 bp, Artificial Sequence
  Synthetic
aacagctcacgcaggccgtg

SEQ ID NO: 27 - DNA, 20 bp, Artificial Sequence
  Synthetic
gcagtcccagcgcggcgagg

SEQ ID NO: 28 - DNA, 20 bp, Artificial Sequence
  Synthetic
cgaatgtcgacccggcatgt

SEQ ID NO: 29 - DNA, 20 bp, Artificial Sequence
  Synthetic
ggtcgacattcgcagacacg

SEQ ID NO: 30 - DNA, 20 bp, Artificial Sequence
  Synthetic
cccgtgtctgcgaatgtcga

SEQ ID NO: 31 - DNA, 20 bp, Artificial Sequence
  Synthetic
cgaggtcgtctaagggacac

SEQ ID NO: 32 - DNA, 20 bp, Artificial Sequence
  Synthetic
cacgaggtcgtctaagggac

SEQ ID NO: 33 - DNA, 20 bp, Artificial Sequence
  Synthetic
ccgcacctgcgaatgtcgac

SEQ ID NO: 34 - DNA, 20 bp, Artificial Sequence
  Synthetic
ttctcgaggtaagggcctcg

SEQ ID NO: 35 - DNA, 20 bp, Artificial Sequence
  Synthetic
cgaagggtcgatcctggcag

SEQ ID NO: 36 - DNA, 20 bp, Artificial Sequence
  Synthetic
gcgtgacttgttctcgaggt

SEQ ID NO: 37 - DNA, 20 bp, Artificial Sequence
  Synthetic
tacctcgagaacaagtcacg

SEQ ID NO: 38 - DNA, 20 bp, Artificial Sequence
  Synthetic
gcgaatgtcgacctggcatg

SEQ ID NO: 39 - DNA, 20 bp, Artificial Sequence
  Synthetic
ctaaactgacgagggctccc

SEQ ID NO: 40 - DNA, 20 bp, Artificial Sequence
  Synthetic
tacctcgagaacagctcacg

SEQ ID NO: 41 - DNA, 20 bp, Artificial Sequence
  Synthetic
gcgtgagctgttctcgaggt

SEQ ID NO: 42 - DNA, 20 bp, Artificial Sequence
  Synthetic
acctcgagaacaagtcacgc

SEQ ID NO: 43 - DNA, 20 bp, Artificial Sequence
  Synthetic
gtccaggtagtagtcgggct

SEQ ID NO: 44 - DNA, 20 bp, Artificial Sequence
  Synthetic
caggtaagcgggtgacgggc

SEQ ID NO: 45 - DNA, 20 bp, Artificial Sequence
  Synthetic
ttagacgacctcgtggagaa

SEQ ID NO: 46 - DNA, 20 bp, Artificial Sequence
  Synthetic
cggggatgcgcgtcctcctc

SEQ ID NO: 47 - DNA, 20 bp, Artificial Sequence
  Synthetic
cgagaacaagtcacgcaggc

SEQ ID NO: 48 - DNA, 20 bp, Artificial Sequence
  Synthetic
cgtggagaagacgccgtgtg

SEQ ID NO: 49 - DNA, 20 bp, Artificial Sequence
  Synthetic
gttctcgaggtaagggcctt

SEQ ID NO: 50 - DNA, 20 bp, Artificial Sequence
  Synthetic
acctgccaggatcgaccctt

SEQ ID NO: 51 - DNA, 20 bp, Artificial Sequence
  Synthetic
cacctgcgaatgtcgacctg

SEQ ID NO: 52 - DNA, 20 bp, Artificial Sequence
  Synthetic
tctgtccggcagggatgatt

SEQ ID NO: 53 - DNA, 20 bp, Artificial Sequence
  Synthetic
cacggcctgcgtgagctgtt

SEQ ID NO: 54 - DNA, 20 bp, Artificial Sequence
  Synthetic
cctcgccgcgctgggactgc

SEQ ID NO: 55 - RNA, 20 nt, Artificial Sequence
  Synthetic
acaugccgggucgacauucg

SEQ ID NO: 56 - RNA, 20 nt, Artificial Sequence
  Synthetic
cgugucugcgaaugucgacc

SEQ ID NO: 57 - RNA, 20 nt, Artificial Sequence
  Synthetic
ucgacauucgcagacacggg

SEQ ID NO: 58 - RNA, 20 nt, Artificial Sequence
  Synthetic
gugucccuuagacgaccucg

SEQ ID NO: 59 - RNA, 20 nt, Artificial Sequence
  Synthetic
gucccuuagacgaccucgug

SEQ ID NO: 60 - RNA, 20 nt, Artificial Sequence
  Synthetic
gucgacauucgcaggugcgg

SEQ ID NO: 61 - RNA, 20 nt, Artificial Sequence
  Synthetic
cgaggcccuuaccucgagaa

SEQ ID NO: 62 - RNA, 20 nt, Artificial Sequence
  Synthetic
cugccaggaucgacccuucg

SEQ ID NO: 63 - RNA, 20 nt, Artificial Sequence
  Synthetic
accucgagaacaagucacgc

SEQ ID NO: 64 - RNA, 20 nt, Artificial Sequence
  Synthetic
cgugacuuguucucgaggua

SEQ ID NO: 65 - RNA, 20 nt, Artificial Sequence
  Synthetic
caugccaggucgacauucgc

SEQ ID NO: 66 - RNA, 20 nt, Artificial Sequence
  Synthetic
gggagcccucgucaguuuag

SEQ ID NO: 67 - RNA, 20 nt, Artificial Sequence
  Synthetic
cgugagcuguucucgaggua

SEQ ID NO: 68 - RNA, 20 nt, Artificial Sequence
  Synthetic
accucgagaacagcucacgc

SEQ ID NO: 69 - RNA, 20 nt, Artificial Sequence
  Synthetic
gcgugacuuguucucgaggu

SEQ ID NO: 70 - RNA, 20 nt, Artificial Sequence
  Synthetic
agcccgacuacuaccuggac

SEQ ID NO: 71 - RNA, 20 nt, Artificial Sequence
  Synthetic
gcccgucacccgcuuaccug

SEQ ID NO: 72 - RNA, 20 nt, Artificial Sequence
  Synthetic
uucuccacgaggucgucuaa

SEQ ID NO: 73 - RNA, 20 nt, Artificial Sequence
  Synthetic
gaggaggacgcgcauccccg

SEQ ID NO: 74 - RNA, 20 nt, Artificial Sequence
  Synthetic
gccugcgugacuuguucucg

SEQ ID NO: 75 - RNA, 20 nt, Artificial Sequence
  Synthetic
cacacggcgucuucuccacg

SEQ ID NO: 76 - RNA, 20 nt, Artificial Sequence
  Synthetic
aaggcccuuaccucgagaac

SEQ ID NO: 77 - RNA, 20 nt, Artificial Sequence
  Synthetic
aagggucgauccuggcaggu

SEQ ID NO: 78 - RNA, 20 nt, Artificial Sequence
  Synthetic
caggucgacauucgcaggug

SEQ ID NO: 79 - RNA, 20 nt, Artificial Sequence
  Synthetic
aaucaucccugccggacaga

SEQ ID NO: 80 - RNA, 20 nt, Artificial Sequence
  Synthetic
aacagcucacgcaggccgug

SEQ ID NO: 81 - RNA, 20 nt, Artificial Sequence
  Synthetic
gcagucccagcgcggcgagg

SEQ ID NO: 82 - DNA, 21 bp, Artificial Sequence
  Synthetic
gacggctggacactcacccac

SEQ ID NO: 83 - DNA, 21 bp, Artificial Sequence
  Synthetic
tgacctgccaggatcgaccct

SEQ ID NO: 84 - DNA, 21 bp, Artificial Sequence
  Synthetic
catgacaggtgtcctcgaagg

SEQ ID NO: 85 - DNA, 21 bp, Artificial Sequence
  Synthetic
aggcgccacccctggggaccc

SEQ ID NO: 86 - DNA, 21 bp, Artificial Sequence
  Synthetic
ccagggctgttcccgacacag

SEQ ID NO: 87 - DNA, 21 bp, Artificial Sequence
  Synthetic
gaagcagcttcctgggcgagg

SEQ ID NO: 88 - DNA, 21 bp, Artificial Sequence
  Synthetic
agagctccacctgtgctaccc

SEQ ID NO: 89 - DNA, 21 bp, Artificial Sequence
  Synthetic
cccagggctgttcccgacaca

SEQ ID NO: 90 - DNA, 21 bp, Artificial Sequence
  Synthetic
gcagattccagacccaggagg

SEQ ID NO: 91 - DNA, 21 bp, Artificial Sequence
  Synthetic
tgcaatcaccagacccctctg

SEQ ID NO: 92 - DNA, 21 bp, Artificial Sequence
  Synthetic
gtgggtgagtgtccagccgtc

SEQ ID NO: 93 - DNA, 21 bp, Artificial Sequence
  Synthetic
agggtcgatcctggcaggtca

SEQ ID NO: 94 - DNA, 21 bp, Artificial Sequence
  Synthetic
ccttcgaggacacctgtcatg

SEQ ID NO: 95 - DNA, 21 bp, Artificial Sequence
  Synthetic
gggtccccaggggtggcgcct

SEQ ID NO: 96 - DNA, 21 bp, Artificial Sequence
  Synthetic
ctgtgtcgggaacagccctgg

SEQ ID NO: 97 - DNA, 21 bp, Artificial Sequence
  Synthetic
cctcgcccaggaagctgcttc

SEQ ID NO: 98 - DNA, 21 bp, Artificial Sequence
  Synthetic
gggtagcacaggtggagctct

SEQ ID NO: 99 - DNA, 21 bp, Artificial Sequence
  Synthetic
tgtgtcgggaacagccctggg

SEQ ID NO: 100 - DNA, 21 bp, Artificial Sequence
  Synthetic
cctcctgggtctggaatctgc

SEQ ID NO: 101 - DNA, 21 bp, Artificial Sequence
  Synthetic
cagaggggtctggtgattgca

SEQ ID NO: 102 - RNA, 21 nt, Artificial Sequence
  Synthetic
gacggcuggacacucacccac

SEQ ID NO: 103 - RNA, 21 nt, Artificial Sequence
  Synthetic
ugaccugccaggaucgacccu

SEQ ID NO: 104 - RNA, 21 nt, Artificial Sequence
  Synthetic
caugacagguguccucgaagg

SEQ ID NO: 105 - RNA, 21 nt, Artificial Sequence
  Synthetic
aggcgccaccccuggggaccc

SEQ ID NO: 106 - RNA, 21 nt, Artificial Sequence
  Synthetic
ccagggcuguucccgacacag

SEQ ID NO: 107 - RNA, 21 nt, Artificial Sequence
  Synthetic
gaagcagcuuccugggcgagg

SEQ ID NO: 108 - RNA, 21 nt, Artificial Sequence
  Synthetic
agagcuccaccugugcuaccc

SEQ ID NO: 109 - RNA, 21 nt, Artificial Sequence
  Synthetic
cccagggcuguucccgacaca

SEQ ID NO: 110 - RNA, 21 nt, Artificial Sequence
  Synthetic
gcagauuccagacccaggagg

SEQ ID NO: 111 - RNA, 21 nt, Artificial Sequence
  Synthetic
ugcaaucaccagaccccucug

SEQ ID NO: 112 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 113 - DNA, 3760 bp, Homo sapiens
ctgagtcatctctgcacgtgtttgcccccttttttcttcgctgcttgtagctaagtgttcctggaaccaatttgatacgggagaactaaggctgaaacctcggaggaacaaccacttttgaagtgacttcgcggcgtgcgttgggtgcggactaggtggccgcggcgggagtgtgctggagcctgaagtccacgcgcgcggctgagaaccgccgggaccgcacgtgggcgccgcgcgcttcccccgcttcccaggtgggcgccggccgccaggccacctcacgtccggccccggggatgcgcgtcctcctcgccgcgctgggactgctgttcctgggggcgctacgagccttcccacaggatcgacccttcgaggacacctgtcatggaaaccccagccactactatgacaaggctgtcaggaggtgctgttaccgctgccccatggggctgttcccgacacagcagtgcccacagaggcctactgactgcaggaagcagtgtgagcctgactactacctggatgaggccgaccgctgtacagcctgcgtgacttgttctcgagacgacctcgtggagaagacgccgtgtgcatggaactcctcccgtgtctgcgaatgtcgacccggcatgttctgttccacgtctgccgtcaactcctgtgcccgctgcttcttccattctgtctgtccggcagggatgattgtcaagttcccaggcacggcgcagaagaacacggtctgtgagccggcttccccaggggtcagccctgcctgtgccagcccagagaactgcaaggaaccctccagtggcaccatcccccaggccaagcccaccccggtgtccccagcaacctccagtgccagcaccatgcctgtaagagggggcacccgcctcgcccaggaagctgcttctaaactgacgagggctcccgactctccctcctctgtgggaaggcctagttcagatccaggtctgtccccaacacagccatgcccagaggggtctggtgattgcagaaagcagtgtgagcccgactactacctggacgaggccggccgctgcacggcctgcgtgagctgttctcgagatgaccttgtggagaagacgccatgtgcatggaactcctcccgcacctgcgaatgtcgacctggcatgatctgtgccacatcagccaccaactcctgtgcccgctgtgtcccctacccaatctgtgcagcagagacggtcaccaagccccaggatatggctgagaaggacaccacctttgaggcgccacccctggggacccagccggactgcaaccccaccccagagaatggcgaggcgcctgccagcaccagccccactcagagcttgctggtggactcccaggccagtaagacgctgcccatcccaaccagcgctcccgtcgctctctcctccacggggaagcccgttctggatgcagggccagtgctcttctgggtgatcctggtgttggttgtggtggtcggctccagcgccttcctcctgtgccaccggagggcctgcaggaagcgaattcggcagaagctccacctgtgctacccggtccagacctcccagcccaagctagagcttgtggattccagacccaggaggagctcaacgcagctgaggagtggtgcgtcggtgacagaacccgtcgcggaagagcgagggttaatgagccagccactgatggagacctgccacagcgtgggggcagcctacctggagagcctgccgctgcaggatgccagcccggccgggggcccctcgtcccccagggaccttcctgagccccgggtgtccacggagcacaccaataacaagattgagaaaatctacatcatgaaggctgacaccgtgatcgtggggaccgtgaaggctgagctgccggagggccggggcctggcggggccagcagagcccgagttggaggaggagctggaggcggaccataccccccactaccccgagcaggagacagaaccgcctctgggcagctgcagcgatgtcatgctctcagtggaagaggaagggaaagaagaccccttgcccacagctgcctctggaaagtgaggcctgggctgggctggggctaggagggcagcagggtggcctctgggaggccaggatggcactgttggcaccgaggttgggggcagaggcccatctggcctgaactgaggctccagcatctagtggtggaccggccggtcactgcaggggtctggtggtctctgcttgcatccccaacttagctgtcccctgacccagagcctaggggatccggggcttgtacagaagagacagtccaaggggactggatcccagcagtgatgttggttgaggcagcaaacagatggcaggatgggcactgccgagaacagcattggtcccagagccctgggcatcagaccttaaccaccaggcccacagcccagcgagggagaggtcgtgaggccagctcccggggcccctgtaaccctactctcctctctccctggacctcagaggtgacacccattgggcccttccggcatgcccccagttactgtaaatgtggcccccagtgggcatggagccagtgcctgtggttgtttctccagagtcaaaagggaagtcgagggatggggcgtcgtcagctggcactgtctctgctgcagcggccacactgtactctgcactggtgtgagggcccctgcctggactgtgggaccctcctggtgctgcccaccttccctgtcctgtagccccctcggtgggcccagggcctagggcccaggatcaagtcactcatctcagaatgtccccaccaatccccgccacagcaggcgcctcgggtcccagatgtctgcagccctcagcagctgcagaccgcccctcaccaacccagagaacctgctttactttgcccagggacttcctccccatgtgaacatggggaacttcgggccctgcctggagtccttgaccgctctctgtgggccccacccactctgtcctgggaaatgaagaagcatcttccttaggtctgccctgcttgcaaatccactagcaccgaccccaccacctggttccggctctgcacgctttggggtgtggatgtcgagaggcaccacggcctcacccaggcatctgctttactctggaccataggaaacaagaccgtttggaggtttcatcaggattttgggtttttcacatttcacgctaaggagtagtggccctgacttccggtcggctggccagctgactccctagggccttcagacgtgtatgcaaatgagtgatggataaggatgagtcttggagttgcgggcagcctggagactcgtggacttaccgcctggaggcaggcccgggaaggctgctgtttactcatcgggcagccacgtgctctctggaggaagtgatagtttctgaaaccgctcagatgttttggggaaagttggagaagccgtggccttgcgagaggtggttacaccagaacctggacattggccagaagaagcttaagtgggcagacactgtttgcccagtgtttgtgcaaggatggagtgggtgtctctgcatcacccacagccgcagctgtaaggcacgctggaaggcacacgcctgccaggcagggcagtctggcgcccatgatgggagggattgacatgtttcaacaaaataatgcacttccttacctagtggcccttcacacaacttttgaatctctaaaaatccataaaatccttaaagaactgtaa

SEQ ID NO: 114 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 115 - protein, 24 aa, Artificial Sequence
  Synthetic
IYIWAPLAGTCGVLLLSLVITLYC

SEQ ID NO: 116 - protein, 68 aa, Artificial Sequence
  Synthetic
FWVLVVVGGVLACYSLLVTVAFIIFWVRSKRSRLLHSDYMNMTPRRPGPTRKHYQPYAPPRDFAAYRS

SEQ ID NO: 117 - protein, 41 aa, Artificial Sequence
  Synthetic
KRGRKKLLYIFKQPFMRVQTTQEEDGCSCRFPEEEEGGCEL

SEQ ID NO: 118 - protein, 113 aa, Artificial Sequence
  Synthetic
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR